(19) United States
(12) Patent Application Publication
(10) Pub. No.: US 2022/0235105 A1
(43) Pub. Date: Jul. 28, 2022

(54) METHODS AND COMPOSITIONS FOR VISUALIZING SUMO

(71) Applicant: College of William & Mary, Williamsburg, VA (US)

(72) Inventors: Oliver Kerscher, Williamsburg, VA (US); Rui Yin, Williamsburg, VA (US)

(73) Assignee: College of William & Mary, Williamsburg, VA (US)

(21) Appl. No.: 17/712,706

(22) Filed: Apr. 4, 2022

Related U.S. Application Data
(62) Division of application No. 16/744,889, filed on Jan. 16, 2020.
(60) Provisional application No. 62/793,709, filed on Jan. 17, 2019.

(51) Int. Cl. C07K 14/47; C07K 14/435; G01N 33/574; C12N 15/85

(52) U.S. Cl. CPC C07K 14/4702; C07K 14/43595; A61K 38/00; C12N 15/85; C07K 2319/20; G01N 33/57484

(57) ABSTRACT

The present disclosure describes pan-SUMO trapping proteins and fusion proteins comprising the pan-SUMO trapping proteins that are stable and bind SUMO-modified proteins with high avidity. The proteins described herein can be used to detect the localization of SUMO-modified proteins cells. The proteins described herein can be used to identify biomarkers for diseases associated with oxidative stress. They can also be used to diagnose and monitor diseases associated with genotoxic and/or proteotoxic stress conditions.

### CROSS REFERENCE TO RELATED APPLICATIONS

This application is a divisional of U.S. patent application Ser. No. 16/744,889 filed Jan. 16, 2020, which claims the benefit of U.S. Provisional Patent Application No. 62/793,709, filed on Jan. 17, 2019, which are hereby incorporated by reference in their entirety.

### SEQUENCE LISTING INFORMATION

A computer readable textfile, entitled “0267-0002US_ST25.bd”, created on or about Jan. 7, 2020 with a file size of about 10 KB, contains the sequence listing for this application and is hereby incorporated by reference in its entirety.

### TECHNICAL FIELD

The present disclosure describes products and compositions for binding ubiquitin-like modifier (SUMO) proteins and methods of using the products and compositions to detect SUMO proteins, including SUMO conjugates, and for diagnosing and monitoring diseases.

### BACKGROUND

SUMO is an essential and highly conserved, small ubiquitin-like modifier protein. SUMO is a signaling protein that becomes attached to other cellular proteins and frequently serves as a stress signal for cells that experience infections, protein misfolding, or DNA damage associated with cancerous transformation. Indeed, evidence is mounting that SUMO, proteins involved in SUMO dynamics, and certain SUMO-modified proteins are grossly increased or mislocalized in some diseases and may represent useful biomarkers in biomedical research and the diagnosis of cancer, heart diseases, viral infection, fertility, and neurodegenerative diseases. Depending on growth conditions, cells may contain hundreds or thousands of proteins that are modified with SUMO or SUMO chains (for review see Kerscher, 2016). This represents a considerable difficulty for the functional analyses of specific SUMO-modified proteins, especially since only a fraction of a potential sumoylation target is actually modified (Hay, 2005). Next to their role in essential cellular processes such as transcriptional regulation, protein homeostasis, the response to cellular stress, and chromatin remodeling during mitosis and meiosis; it has now become apparent that SUMO, SUMO-modified proteins, and SUMO pathway components also have potential as biomarkers for pathologies such as cancer and neurodegenerative disorders. This underscores the need for robust, reliable, and readily available tools and innovative approaches for the detection and functional analysis of SUMO-modified proteins in a variety of cells and samples.

In many systems, SUMO-specific antibodies are the reagents of choice for the detection, isolation and functional analyses of SUMO-modified proteins (Pelisch et al., 2017; X.-D. Zhang et al., 2008). However, some commercially available SUMO-specific antibodies are expensive, limited in quantity or availability, may exhibit wildly variable affinities and cross-reactivity, and in some instances lack in reproducibility (Baker, 2015). A related approach is the expression of epitope-tagged SUMO in transformed cells and organisms but linking epitope tags to SUMO may artificially lower its conjugation to protein targets (Z. Wang & Prelich, 2009). Additionally, epitopes are not useful when untransformed cells or tissues are evaluated.

Accordingly, there is a need to develop a reliable and cost-effective reagent for detecting SUMO proteins.

### SUMMARY

This Summary is provided to introduce a selection of concepts in a simplified form that are further described below in the Detailed Description. This Summary is not intended to identify all key features or essential features of the claimed subject matter, nor is it intended to be used alone as an aid in determining the scope of the claimed subject matter.

The present disclosure describes a pan-SUMO trapping protein that binds SUMO-modified proteins, such as SUMO conjugates and SUMO chains, with high avidity and exhibits enhanced stability. Examples of pan-SUMO trapping proteins that bind SUMO-modified proteins include Ulp variants, such as UTAG. In embodiments, the pan-SUMO trapping protein is KmUTAG, which is a Ulp variant that bind SUMO proteins with high avidity and exhibit enhanced stability as compared to ScUTAG.

The present disclosure describes a fusion protein including a pan-SUMO trapping protein and a fluorescent protein (fl). In embodiments, the pan-SUMO trapping protein is KmUTAG, and the fusion protein is KmUTAG-fl. Various fluorescent proteins can be used for making the fusion protein described herein. Examples include mCherry, mPlum, mRaspberry, HcRed-Tandem, mRFP1, and the like.

The present disclosure also describes methods of preparing the proteins and fusion proteins described herein. In embodiments, the proteins and fusion proteins can be prepared by recombinant means.

The present disclosure also describes methods of using the proteins and fusion protein described herein for visualizing and/or localizing SUMO proteins in different types of cells. Examples of the different types of cells include mammalian tissue culture cells and nematode oocytes. In embodiments, the fusion protein described herein can be used to detect SUMO proteins in mammalian cancer cells, such as prostate cancer cells. Additionally, the present disclosure describes methods of using the proteins and fusion protein described herein to detect cells that are under oxidative stress. In embodiments, the proteins and fusion proteins described herein can be used to diagnose and monitor diseases associated with cancer and oxidative stress.

### BRIEF DESCRIPTION OF THE DRAWINGS

FIG. 1 shows a schematic representation of KmUTAG-flmc, a fusion protein including KmUTAG and the fluorescent protein mCherry, which is one embodiment of a KmUTAG-fl fusion protein.

FIGS. 2A-2D show confocal microscopy images of PNT2 cells grown on coverslips, fixed, and stained before applying mounting media. Slides were visualized using appropriate filters for the staining agents.

FIGS. 3A-3E show developing C. elegans oocytes stained with either the anti-SUMO antibody SUMO 6F2 (FIGS. 3A, 3B, and 3C) or KmUTAG-fl (FIGS. 3D and 3E).

FIGS. 4A-4D show SUMO enrichment of UV irradiated PC3 and PNT2 cells. (A) UV dosage changed the SUMO profile of PC3 cells but not of PNT2 cells. UV irradiated PC3 and PNT2 cells (0 (Control), 50 mJ/m², and 150 mJ/m²) were stained with KmUTAG-fl (SUMO) and visualized using confocal microscopy for mCherry (KmUTAG-fl, red) and DAPI (blue). (B) Nuclear and cytosolic KmUTAG-fl signal intensity in PC3 and PNT2 cells were quantified with CellProfiler [Carpenter et al., 2006 PMID: 17076895]. PC3: 0 mJ/m², n=54; 50 mJ/m², n=27; 150 mJ/m², n=68. PNT2: 0 mJ/m², n=20; 50 mJ/m², n=20; P 150 mJ/m², n=29. (C) Increased cytosolic SUMO accumulation (KmUTAG-fl signal intensity) of PC3 cells compared to PNT2 cells. Intensity change was calculated as the difference between the UV irradiated and untreated Control cells. (D) Relative Cytosolic Enrichment (RCE) ratio of SUMO (KmUTAG-fl signal) in PC3 cells increased with UV dosage. The RCE was calculated as the ratio between cytosolic and nuclear fluorescence intensity. Statistical analysis by unpaired t-test (NS: P>0.05, *P≤0.05, **P≤0.01, ***P≤0.001). Scale bars: 20 μm.

FIGS. 5A-5C show KmUTAG-fl and Anti-SUMO2 8A2 antibody co-staining of UV irradiated cancer cells. (A) Similar SUMO profiles were observed using the KmUTAG-fl biosensor (red) and the anti-SUMO antibody (green). PC3 cells were irradiated with 250 mJ/m² UV. Fixed cells were co-stained with KmUTAG-fl (SUMO), anti-SUMO2 8A2 antibody (SUMO2), and DAPI (nucleus, blue). Control cells were not UV-irradiated. Scale bars=20 μm. (B) Average cytosolic and nuclear KmUTAG-fl and 8A2 signal intensity (SUMO accumulation) were quantified with CellProfiler (km_Control n=23; km_UV n=22; antiS2_Control n=23; antiS2_UV n=22). AU: arbitrary units. (C) Percent change in cytosolic and nuclear KmUTAG-fl and antibody signal intensity in PC3 cells. Statistical analysis by unpaired t-test (NS: P>0.05, *P≤0.05, **P≤0.01, ***P≤0.001). Scale bars: 20 μm.

FIGS. 6A-6G show SUMO enrichment of H₂O₂ treated PC3 and PNT2 cells. H₂O₂ treated (0.5 μM, 25 μM, 1 mM, 10 mM, 30 mM) or control PC3 cells were fixed and stained for SUMO with KmUTAG-fl, visualized and quantified as previously described. (B) Average cytosolic and nuclear KmUTAG-fl signal intensity (SUMO accumulation) were quantified with CellProfiler (PC3 Control n=47; PC3 0.5 μM n=44, PC3 25 μM n=54; PC3 1 mM n=23; PC3 10 mM n=25; PC3 30 mM n=17) (C) H₂O₂ treated (0.5 mM, 5 mM, 20 mM) or control PNT2 cells were fixed and stained for SUMO with KmUTAG-fl, visualized and quantified as above. (D) Average cytosolic and nuclear KmUTAG-fl (SUMO accumulation) were quantified with CellProfiler PNT2 Control n=24; PNT2 0.5 μM n=27, PNT2 20 μM n=33; PNT2 5 mM n=20). (E) Increased cytosolic and nuclear SUMO accumulation (KmUTAG-fl signal intensity) of PC3 cells compared to PNT2 cells. Intensity change was calculated as the difference between the H₂O₂ irradiated and untreated Control cells. (F) Relative Cytosolic Enrichment (RCE) ratio of SUMO (KmUTAG-fl signal) in PC3 cells increased with H₂O₂ concentration. The RCE was calculated as the ratio between cytosolic and nuclear fluorescence intensity. (G) Variability in the Relative Cytosolic Enrichment (ROE) ratio of SUMO (KmUTAG-fl signal) in PNT2 cells at various H₂O₂ concentrations. The RCE was calculated as the ratio between cytosolic and nuclear fluorescence intensity. Statistical analysis by unpaired t-test (NS: P>0.05, *P≤0.05, **P≤0.01, ***P≤0.001). Scale bars: 20 μm.

FIGS. 7A-7F show recovery from peroxide stress is accompanied by gradually decreasing SUMO level. (A) H₂O₂ treated (1 mM) or control PC3 and PNT2 cells were fixed and stained for SUMO with KmUTAG-fl after the indicated recovery times (0-5 hours). Representative cells samples are shown. Merged cells: DAPI (blue), KmUTAG-fl (red) (B) Same as (A) except PNT2 cells. (C) Decreasing KmUTAG-fl signal intensity (cytosolic and nuclear) in recovering PC3 cells (PC3 0 hr n=54; PC3 1 hr n=60; PC3 2 hr n=93; PC3 3 hr n=15; PC3 4 hr n=51; PC3 5 hr n=54; PC3 Control n=47). (D) Little or no significant change in cytosolic and nuclear KmUTAG-fl signal intensity in recovering PNT2: (0 hr n=72; PNT2 1 hr n=95; PNT2 2 hr n=103; PNT2 3 hr n=60; PNT2 4 hr n=102; PNT2 5 hr n=103; PNT2 Control n=70). (E) Comparison in the change [%] of cytosolic and nuclear (SUMO) KmUTAG-fl signal intensity in PC3 and PNT2 cells. Top panel: change [%] of cytosolic (SUMO) KmUTAG-fl signal. Bottom panel: change [%] of nuclear (SUMO) KmUTAG-fl signal. The intensity change was calculated as the intensity difference between the treated cells and control cells. (F) The Relative Cytosolic Enrichment (RCE) ratio of the KmUTAG-fl signal in PC3 cells (left panel) and PNT2 cells (right panel) after the indicated recovery times. Note the delayed and reduced RCE of PNT2 cells compared to PC3 cells. Statistical analysis by unpaired t-test (NS: P>0.05, *P≤0.05, **P≤0.01, ***P≤0.001). Scale bars: 20 μm.

FIGS. 8A-8D show H₂O₂ stress induces increase in cytosolic SUMO levels in LNCaP cells with low-metastatic potential. A) H₂O₂ treated (1 mM) or Control LNCaP, PC3 and PNT2 cells were fixed and stained for SUMO with KmUTAG-fl. Representative cells of each cell line are shown. Merged cells: DAPI (blue) and KmUTAG-fl (red). (B) KmUTAG-fl signal intensity in nuclei and cytosol of LNCaP, PC3 and PNT2 cells across treatment groups. (LNCaP_Control n=402; LNCaP_Peroxide n=345; PC3_Control n=368; PC3_Peroxide n=422; PNT2_Control n=342; PNT2_Peroxide n=331). (C) The largest change [%] in KmUTAG-fl signal intensity is observed in nuclei and cytosol of LNCaP, PC3 when compared to PNT2 cells. The intensity change was calculated as the intensity difference between the peroxide-treated cells and control cells. (D) The relative cytosolic enrichment (RCE) ratio of the KmUTAG-fl signal is most pronounced in LNCaP, PC3 when compared to PNT2 cells. Statistical analysis by unpaired t-test (NS: P>0.05, *P≤0.05, **P≤0.01, ***P≤0.001). Scale bars: 20 μm.

### DETAILED DESCRIPTION

The present disclosure describes novel polypeptides that bind SUMO-modified proteins with high avidity and exhibit enhanced stability, fusion proteins including the novel polypeptides, compositions including the polypeptides and fusion proteins, methods for their production, and methods and systems for using them in detecting SUMO-modified proteins and diagnosing and monitoring diseases and treatments.

The terms “a,” “an,” “the” and similar referents used in the context of describing the claimed subject matter (especially in the context of the claims) are to be construed to cover both the singular and the plural, unless otherwise indicated herein or clearly contradicted by context.

The terms “protein” and “polypeptide” are used interchangeably to refer to compounds comprising amino acids joined via peptide bonds. Amino acids can be naturally or non-naturally occurring. The terms “amino acids,” “amino acid residues,” and “residues” are used interchangeably.

The terms “chimeric protein” and “fusion protein” are used interchangeably to refer to a protein in which different portions of the protein are obtained from different sources such that the entire molecule is not naturally occurring. A chimeric protein includes heterologous polypeptides. A chimeric protein can contain amino acid sequences from the same species or different species as long as they are not arranged together in the same way as they exist in nature. Examples of a chimeric protein include proteins disclosed herein that include one or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, or a polyhistidine tag, e.g. typically four or more histidine amino acids.

The term “codon optimization” refers to a nucleic acid sequence optimized for expression in bacterial or eukaryotic expression host systems. Codon optimized nucleic acid sequences can be referred to as conservatively modified variant nucleic acid sequence because the nucleic acids are optimized using silent mutations or variations using the degeneracy of the genetic code, and they encode a sequence without any amino acid alterations.

The term “variant” refers to a polypeptide having a “desired functional activity” but includes one or more modification or alterations in the polypeptide's amino acid sequence. The “desired functional activity” as used herein refers to “pan-SUMO trapping” activity. The alteration can be an amino acid substitution, insertion, and/or deletion. A substitution refers to the replacement of an amino acid occupying a position with a different amino acid; a deletion refers to the removal of an amino acid occupying a position; and an insertion refers to the addition of an amino acid to its amino acid sequence.

A substitution can be a conservative amino acid substitution. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups are: valine-leucine-isoleucine, phenylalanine-tyrosine, lysine-arginine, alanine-valine, and asparagine-glutamine.

A variant can also include “non-conservative” amino acid changes (e.g., replacement of a glycine with a tryptophan) and retain or improve the desired functional activity. Similar minor variations can also include amino acid deletions or insertions (in other words, additions), or both. Guidance in determining which and how many amino acid residues can be substituted, inserted or deleted without abolishing the desired functional activity may be found using computer programs well known in the art, for example, DNAStar software. Variants can be tested in functional assays. Certain variants have less than 10%, less than 5%, or less than 2% changes (whether substitutions, deletions, and so on).

The term “derivative” refers to a structurally similar polypeptide that retains sufficient functional attributes of an original polypeptide. The derivative can be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, replacing a oxygen atom with a sulfur atom, or replacing an amino group with a hydroxyl group. A derivative can be two or more polypeptides linked together by a linking group. The linking group can be biodegradable. Derivatives can be prepared by any variety of synthetic methods or appropriate adaptations presented in synthetic or organic chemistry text books.

The term “sequence identity” refers to the relatedness between two amino acid sequences or two nucleic acid sequences when the two sequences are aligned. The term “percent sequence identity” refers to the percentage of amino acids or bases that are the same in comparing the two sequences. The sequence identity between two amino acid sequences or between two nucleic acid sequences can be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), e.g., version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the --nobrief option) is used as the percent identity and is calculated as follows:

(Identical Residues×100)/(Length of Alignment−Total Number of Gaps in Alignment)

or

(Identical nucleotides×100)/(Length of Alignment−Total Number of Gaps in Alignment).

Other suitable software programs for sequence alignment include those described in CURRENT PROTOCOLS IN MOLECULAR BIOLOGY (F. M. Ausubel et al. (eds) 1987, Supplement 30, section 7.7.18). Other examples include the GCG Pileup program; FASTA (Pearson et al. (1988) Proc. Natl, Acad. Sci USA 85:2444-2448); BLAST (BLAST Manual, Altschul et al., Nat'l Cent. Biotechnol. Inf., Natl Lib. Med. (NCIB NLM NIH), Bethesda, Md., and Altschul et al., (1997) NAR 25:3389-3402); ALIGN Plus (Scientific and Educational Software, Pa.) using default parameters; and TFASTA Data Searching Program available in the Sequence Software Package Version 6.0 (Genetics Computer Group, University of Wisconsin, Madison, Wis.).

The term “SUMO-modified protein” refers to a protein that has undergone sumoylation in which a SUMO protein becomes covalently linked to a cellular protein or to itself to form a SUMO conjugate or SUMO chain.

The term “sumolyation” refers to a reversible posttranslation modification of cellular proteins with SUMO (small ubiquitin-like modifier protein). Sumoylation is a fundamental protein modification process that is conserved from yeast to humans and controls the function, activation, localization, interaction, and half-life of specific cellular proteins. Sumoylation is involved in transcription, DNA repair, chromatin remodeling, splicing, assembly of ribosomes, and other cellular processes. During sumoylation, SUMO proteins covalently attach to other cellular proteins to form SUMO conjugates and SUMO chains. The genes that promote sumoylation and SUMO dynamics require a cascade of SUMO activating (E1), conjugating E2, and ligase enzymes (E3) as well as SUMO proteases and SUMO-targeted ubiquitin ligases (Kerscher, Felberbaum, & Hochstrasser, 2006). A recent study identified more than 4,300 sumoylation sites in more than 1,600 proteins from HeLa cells. A large number of human disease proteins were found to be targets of SUMO modification (Hendriks et al., 2014; Sarge & Park-Sarge, 2009).

The accumulation of sumoylated proteins in the cells is counterbalanced by dedicated SUMO-specific cysteine proteases that cleave SUMO off proteins that have been sumoylated. An example of such a protease is Ulp1. Ulp1 was originally found in budding yeast Saccharomyces cerevisiae (Sc). ScUlp1 is required for processing the SUMO precursor and several nuclear and cytosolic SUMO-modified proteins. Ulp1 is evolutionarily conserved in the form of six distinct SENP (sentrin/SUMO-specific proteases) in mammalian cells. SENP1 and SENP2 are most similar to Ulp1. SENP proteases play a role in ribosome biogenesis and regulate several important nuclear activities including transcription, genome maintenance, recombination, and chromosome segregation. It has been shown that clinically relevant dysregulation or overexpression of the SUMO protease SENP1 plays a role in cancer development and that SENP1 and SENP2 prevent apoptosis of neuronal cells in cell culture and animal models.

Mammalian SENPs have a highly conserved C-terminal catalytic domain. Six SUMO proteases have been found in humans. They are named SENP1-3 and SENP5-7. Similar to the Ulps in yeast, the SENPs contain a C-terminal domain having catalytic activity and a N-terminal domain that regulates cell localization and substrate specificity. SENP1 and SENP2 are most similar to Ulp1.

Human SENP1 is a 644 amino acid long polypeptide having a molecular weight of 73 kDa. Representative amino acid sequences for human SENP1 can be found at accession numbers Q9POU3 (GI: 215273882) and NP_001254523.1 (GI: GI: 390131988). The enzyme commission (EC) number for SENP1 in human is EC 3.4.22.B70 which identify it as a member of the superfamily of cysteine proteases containing a catalytic triad with the following three amino acids: a cysteine at position 602, a histidine at position 533, and an aspartic acid at position 550. Mammalian SENP1s are localized mainly in the nucleus, although it has also been found in the cytosol of some cells.

Human SENP2 is a 589 amino acid long polypeptide having a molecular weight of 67.9 kDa. Representative amino acid sequences for human SENP2 can be found at accession numbers Q9HC62.3 (GI: 143811458) and NP_067640.2 (GI: 54607091).

SUMO proteases such as Ulp1 are highly conserved among yeasts and contain a hallmark carboxy-terminal catalytic Ulp1 Domain (UD). The UD domain transiently binds SUMO-modified proteins and catalyzes desumoylation. It was found that replacing cysteine 580 (C580) in the UD domain of Ulp1 with serine not only renders the UD domain non-catalytic but also traps sumolyated proteins. For simplicity, the catalytic domain of Ulp1 is referred to as “UD,” and the UD of pan-SUMO trapping Ulp1 including C580S mutation is referred to as “UTAG (short for UD TAG).” Accordingly, the UTAG including the C580 mutation derived from Saccharomyces cerevisiae is ScUTAG.

The term “mutant ScUD” refers to a modified UD derived from Sc, such as ScUTAG, which can comprise a C580S mutation. It is important to note that UD by definition is catalytically active (see for example, paragraph [0039] below)

A wild type (WT) UD domain (WT UD) is from a naturally occurring source, such as from Saccharomyces cerevisiae is ScUD, which does not have a mutation in its UD domain. Although SENP1 and SENP2 are referred to as variants of Ulp1, they are also WT proteins and contain a WT UD.

The term “UD equivalent domain” refers to a domain having the same equivalent functional activity as the UD domain of another SUMO protease, for example a Ulp1 ortholog (such as ScUD). The “UD equivalent domain” is the domain for binding and processing SUMO-modified proteins.

UTAG is a pan-SUMO trapping (binding) protein and is catalytically inactive as a protease. It has been shown to be a useful alternative to anti-SUMO antibodies used for the isolation and detection of SUMO-modified proteins. It specifically recognizes natively-folded, conjugated SUMO and not just one or several SUMO epitopes. The discovery of UTAG was based on the finding that a mutation of the catalytic cysteine (C580S) in the Ulp1's SUMO processing UD domain not only prevents SUMO cleavage but also traps SUMO-conjugated proteins with high avidity (Elmore et al., 2011). The term “pan-SUMO trapping protein” refers to a protein that binds SUMO-modified proteins and are stress-tolerant. Pan-SUMO trapping proteins bind SUMO-modified protein with an avidity that is at least 200-fold stronger than the binding of a SUMO interacting motif (SIM) with a SUMO-modified protein. Proteins and protein domains may contain SUMO-interacting motifs (SIMs), which interact non-covalently with SUMO-modified protein. SIMs are characterized by a loose consensus sequence and contain several hydrophobic residues, for example, V/I-X-V/I-V/I (where x is any amino acid). SIMs are embedded in the groove formed between the α-helix and the β-strand of SUMO. The affinities of SIMs for SUMO-modified protein are in the 2-3 μM range.

In embodiments, the “pan-SUMO trapping protein” binds SUMO-modified proteins with high avidity.

The term avidity refers to how strongly an antibody or a similar protein binds to an antigen. Specific antibodies bind their antigens with high avidity and this binding is in the nanomolar range. The term “high avidity” refers to binding of molecules with nanomolar affinity.

The term “stress-tolerant” refers to a molecule that is resistant to stressful conditions, such as elevated temperatures, reducing agents, denaturants, oxidizing agents, and non-ionic detergents, and/or pro-longed incubation time. Elevated temperatures include temperatures of up to at least 42° C. Pro-longed incubation time depends on the extract, temperature, and/or buffers use. Pro-longed incubation includes incubation for greater 12 hours. A stress-tolerant protein is stable under stressful conditions. The stability of a stress-tolerant protein can be compared with its corresponding WT protein.

The present disclosure describes pan-SUMO trapping proteins that bind SUMO-modified proteins with high avidity and are stress-tolerant. Examples of pan-SUMO trapping proteins described herein include Ulp1 variants, variants of the UD domain of Ulp1s, and proteins comprising variants of the UD domain of Ulp1s. Ulp1 variants can be naturally occurring variants obtained from eukaryotic cells including various strains of yeasts and various mammalian cells. As an example, naturally occurring Ulp1 variants from other yeast strains include Ulp1 obtained from Kluyveromyces marxianus (km) or other yeast strains, and naturally occurring Ulp1 variants from mammalian cells include SENP1 and SENP2. The Ulp1 variants can also include non-naturally occurring variants such as those that have been modified or mutated to have the desired functional activity. In embodiments, the pan-SUMO trapping proteins include Ulps of yeasts (such as Km), SENP1, SENP1 variants, SENP2, SENP2 variants, the UD equivalent domain in SENP1 and SENP1 variants, the UD equivalent domain in SENP2 and SENP2 variants, and proteins including the UD equivalent domain in SENP1, SENP2, or variants of SENP1 or SENP2, and they bind SUMO-modified proteins with high avidity and are stress-tolerant.

As an example, the inventors generated a variant of the stress-tolerant yeast Kluyveromyces marxianus (Km) to provide enhanced stability and binding of SUMO-modified proteins with high avidity (Peek, 2018). This polypeptide is referred to as KmUTAG, which includes a mutation of the catalytic cysteine equivalent to the C580S mutation in the UD domain of the mutant ScUTAG. In Km the catalytic cysteine equivalent to C580 is C517. Therefore, the mutation in Km is C517S. KmUTAG tightly binds SUMO-modified proteins with nanomolar affinity. Additionally, KmUTAG is resistant to elevated temperatures (for example, 42° C.), reducing agents (5 mM TCEP), denaturants (up to 2M UREA), oxidizing agents (0.6% hydrogen peroxide), and non-ionic detergents (Peek et al., 2018). This stress tolerance is beneficial during harsh purification condition and pro-longed incubation times, ensuring its stability and SUMO-trapping activity. The present disclosure describes a pan-SUMO trapping protein comprising KmUTAG. In embodiments, the pan-SUMO trapping protein includes KmUTAG comprising the amino acid sequence as set forth in SEQ ID NO: 2.

Other mutants can be generated from other yeast Ulps or mammalian Ulps (SENPs) by mutating their catalytic cysteine that is equivalent to the C580S mutation in the UD domain of the mutant ScUTAG.

The present disclosure describes pan-SUMO trapping proteins including a UD, UD equivalent domain, or UTAG comprising an amino acid sequence having at least 65% sequence identity with the amino acid sequence of KmUTAG of SEQ ID NO: 2. In embodiments, the amino acid sequence of the UD, UD equivalent domain, or UTAG of the pan-SUMO trapping proteins described herein has at least 65%, 70%, 75%, 80%, 82%, 85%, 86%, 87%, 88%, 89%, 90%, 91%, 92%, 93%, 94%, 95%, 96%, 97%, 98%, 99%, or 100% sequence identity with the amino acid sequence of the UD domain of KmUTAG or SEQ ID NO: 2.

The present disclosure describes fusion proteins including a pan-SUMO trapping protein described herein covalently attached to at least one other protein or peptide, for example a fluorescent protein (fl). Examples of fluorescent proteins include mCherry, mPlum, mRaspberry, HcRed-Tandem, mRFP1, mApple, mRuby, mStrawberry, mTangerine, DsRed-Monomer, TagRFP-T, mOrange, dTomatoTandem, Kusabira Orange, mBanana, TagYFP, TagCFP, mCitrine, mECFP, mTagBFP, and mWasabi.

The fusion proteins described herein can include other proteins or peptides such as a protein tag. The protein tag can be an affinity tag for facilitating purification. Examples of affinity tags include a SPOT tag, polyhistidine peptide, the N-terminal glutathione S-transferase (GST), maltose binding protein (MBP), calmodulin binding protein, and streptavidin/biotin tag. The fusion proteins described herein can include a pan-SUMO trapping protein, a fl, and another protein or peptide, such as an affinity tag. In embodiments, the other proteins or peptide can be covalently attached either to the N-terminus or the C-terminus of the pan-SUMO trapping protein.

The fusion proteins described herein include KmUTAG-fl polypeptides. In embodiments, KmUTAG-fl polypeptides are purified, recombinant, fluorescent SUMO-trapping fusion proteins including 1) a stress-tolerant pan-SUMO trapping protein, for example derived from a mutant KmULP1 gene fragment; 2) a fluorescent protein; and optionally 3) an affinity-tag for purification after overexpression in bacteria. In embodiments, the fusion protein comprises KmUTAG, mCherry, and a SPOT tag. In embodiments, the KmUTAG-fl fusion protein comprises the amino acid sequence as set forth in SEQ ID NO: 4.

The present disclosure also describes nucleic acids encoding the pan-SUMO trapping proteins described herein. The nucleic acids encoding the UD, UD equivalent domain, or UTAG of pan-SUMO trapping proteins described herein have at least 65% sequence identity with the nucleic acid encoding KmUTAG. In embodiments, the nucleic acid encoding the UD, UD equivalent domain, or UTAG of the pan-SUMO trapping proteins described herein have at least 65%, 70%, 75%, 80%, 82%, 85%, 86%, 87%, 88%, 89%, 90%, 91%, 92%, 93%, 94%, 95%, 96%, 97%, 98%, 99%, or 100% sequence identity with the nucleic acid sequence encoding the UD or UD equivalent domain of KmUTAG or SEQ ID NO: 1. In embodiments, the nucleic acid sequence encoding the pan-SUMO trapping protein comprises the nucleic acid sequence as set forth SEQ ID NO: 1.

The pan-SUMO trapping proteins described herein, and their encoding nucleic acids can be obtained by chemical synthesis, by recombinant means, or by isolation and modification from a natural source. Moreover, the nucleic acids encoding pan-SUMO trapping proteins can be prepared using a nucleic acid sequence encoding a WT UTAG or a known pan-SUMO trapping protein by any mutagenesis procedure, such as site-directed mutagenesis, synthetic gene construction, semi-synthetic gene construction, random mutagenesis, or any other known methods. The pan-SUMO trapping proteins can be prepared by recombinant means. In embodiments, the nucleic acid encoding KmUTAG can be obtained from the nucleic acid encoding ScUTAG by mutagenesis and introduced into an expression system to obtain KmUTAG. Examples of expression systems include bacterial host cells, fungal host cells including yeast host cells, mammalian host cells, insect host cells, and cell-free systems.

As an example, the preparation of a pan-SUMO trapping proteins can be achieved by mutating the nucleic acid sequence encoding ScUTAG or KmUTAG, introducing the nucleic acid into a suitable host cell, and expressing the mutated sequence in the host cell to obtain a pan-SUMO trapping protein. Introduction of the nucleic acid into a suitable host cell can be by transformation, transduction, transfection, electroporation, viral infection, or similar well-known methods.

Although fusion proteins described herein can be obtained by chemical synthesis, preparation of fusion proteins by recombinant means is more cost effective, as yields are low for polypeptides. Accordingly, the present disclosure describes nucleic acids for use in preparing the fusion proteins described herein, such as by expressing the nucleic acid in a host cell as described above.

Recombinant expression of nucleic acids also includes cloning the nucleic acid encoding the protein of interest into an expression vector. An expression vector includes the components for transcription and translation of the protein. The present disclosure describes expression vectors including a nucleic acid described herein, a promoter, and transcriptional and translational stop signals. The vector can be a linear or closed circular plasmid. The nucleic acid, such as the nucleic acid encoding KmUTAG is inserted into the expression vector in a manner such that it is operably linked with the promoter and the transcriptional and translational stop signals for expression. The expression vector can also include one or more convenient restriction sites to allow insertion of the nucleic acid of insert. Examples of expression vectors include pEV1, pBR322, pUC19, pACYC177, pACYC184, pUB110, pE194, pTA1060, yeast shuttle vectors, pSpot, pGW1, pDual, pADBM5, and insect cell expression vectors (baculovirus vectors). Selection of expression vector to use depends on the compatibility of the vector with the selected expression system, especially, the host cell into which the expression vector will be introduced.

Host cells include any suitable host for expressing nucleic acids comprising an expression vector as described herein. Examples of host cells include prokaryotic cells, eukaryotic cells, and any transformable microorganism in which expression can be achieved. The host cells can be chosen from eukaryotic or prokaryotic systems, such as for example bacterial cells, (Gram negative or Gram positive), yeast cells, mammalian cells, plant cells, fungal cells, and insect cells. In embodiments, the host cells for expression of the polypeptides include those taught in U.S. Pat. Nos. 6,319,691, 6,277,375, 5,643,570, or 5,565,335, each of which is incorporated by reference in its entirety.

Examples of bacterial host cells include Bacillus, Escherichia coli, Trichoderma reesei, and the like. Specific examples of Bacillus include B. subtilis, B. cereus, B. brevis, B. licheniformis, B. stearothermophilus, B. pumilis, B. amyloliquefaciens, B. clusii, or B. megaterium. Examples of yeast host cells include Saccharomyces (Saccharomyces cerevisiae), Aspergillus (Aspergillus niger), Candida, Kluveromyces (Kluyveromyces lactis), and Pichia (Pichia pastoris). Examples of fungal host cells include the phyla Ascomycota, Basidiomycota, Chytridiomycota, and Zygomycota as well as the Oomycota and all mitosporic fungi. Examples of mammalian cells include Chinese hamster ovary (CHO) cells and human kidney cells (HEK). Examples of insect cells include Sf9 cells.

Cell-free protein expression systems involve the in vitro synthesis of a protein using translation-compatible extracts of whole cells, which include all the components needed for transcription, translation, and post-translational modification.

The present disclosure also describes methods of producing pan-SUMO trapping proteins described herein and fusion proteins described herein. The method includes inserting the nucleic acid encoding a protein described herein, for example a pan-SUMO trapping protein or a fusion protein, into an expression vector; introducing the expression vector into a host cell; cultivating the host cell under conditions that enable expression of the protein described herein. The method can also include recovering the expressed protein from the culture media in which the protein was expressed. Recovering the expressed protein can include purification, for example affinity purification. The method can optionally include storing the purified protein, as the pan-SUMO trapping protein described herein is stable.

In embodiments, the KmUTAG-fl fusion protein is inserted into a bacterial overexpression plasmid, such as pEV1 (www.chromoteck.com) and expressed in a suitable expression strain, such as DE3 E. coli. After induction and affinity purification, the KmUTAG-fl fusion protein can be stored cryogenically.

The present disclosure describes codon optimized nucleic acids encoding the pan-SUMO trapping proteins and fusion proteins described herein. In embodiments, the KmUTAG encoded by SEQ ID NO: 1 and the KmUTAG-mCherry fusion protein is encoded by SEQ ID NO: 3.

Moreover, the present disclosure describes compositions including the pan-SUMO trapping proteins or the fusion proteins described herein, or nucleic acids encoding such proteins. The compositions can further include a carrier. The compositions described herein also include pharmaceutical compositions, in which case the carrier is a pharmaceutically acceptable carrier. Pharmaceutically acceptable compositions of the proteins described herein, especially the fusion proteins, can be used for in vitro and in vivo procedures.

The term “pharmaceutically acceptable” means approved by a regulatory agency of the U.S. Federal or a state government or the EMA (European Medicines Agency) or listed in the U.S. Pharmacopeia Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “carrier” refers to a diluent, adjuvant (e.g., Freund's adjuvant (complete and incomplete)), excipient, or vehicle with which the therapeutic is administered. Carriers can be liquids, such as water and oils, including those of petroleum, animal, vegetable or synthetic origin, such as peanut oil, soybean oil, mineral oil, sesame oil and the like. Sterile liquid such as water is a preferred carrier when the pharmaceutical composition is administered intravenously. Saline solutions and aqueous dextrose and glycerol solutions can also be employed as liquid carriers, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. For the use of other excipients and their use see also “Handbook of Pharmaceutical Excipients”, fifth edition, R. C. Rowe, P. J. Seskey and S. C. Owen, Pharmaceutical Press, London, Chicago. Examples of suitable pharmaceutical carriers are described in “Remington's Pharmaceutical Sciences” by E. W. Martin.

The pan-SUMO trapping proteins described herein can be formulated as nanoparticles and nano-gold particles for delivery into cells. Nanoparticles are submicron-sized particles, ranging from 3-200 nm. Nanoparticles can be made of various materials including polymers, lipids, viruses, and organometallic compounds. Polymers can be used to make polymeric nanoparticles, micelles and dendrimers. Lipids can be used to make liposomes. Viruses can be used to make viral nanoparticles, and organometallic compounds can be used to make nanotubes.

The inventors discovered a novel method for investigating sumoylation of proteins and their subcellular localization. Using the recombinant pan-SUMO trapping protein biosensor, for example the KmUTAG-fl, the inventors were able to detect the subcellular localization of sumoylated proteins in mammalian cells and nematode oocytes. Specifically, they were able to show that SUMO, a predominantly nuclear protein, rapidly accumulates in the cytosol of stress-treated cancer cells.

The pan-Sumo trapping proteins and fusion proteins described herein have several advantages in comparison to antibody-based approaches for the study of SUMO. The proteins are stress-tolerant SUMO-trapping reagents that can be easily produced recombinantly in large quantities. They recognize native SUMO isoforms from many species, and unlike commercially available antibodies, they show reduced affinity for free, unconjugated SUMO. As such, the pan-Sumo trapping proteins provide a useful alternative for traditional antibody staining protocols. The pan-Sumo trapping proteins described herein can be used to specifically detect SUMO conjugates in fixed cells. Unlike most antibodies, the pan Sumo trapping proteins, such as KmUTAG-fl, does not require a secondary antibody for visualization, and stained cells are readily visible, for example, under the fluorescence microscope or other detection methods.

The KmUTAG-fl generated by the inventors is a representative stress-tolerant pan-SUMO specific reagents that recognize and trap SUMO-modified proteins. Since SUMO's tertiary structure is highly conserved, SUMO variants from many different systems can be analyzed using the KmUTAG-fl reagents. KmUTAG-fl also detects SUMO orthologs in eukaryotic genetic model systems such as yeast and worms. Accordingly, KmUTAG-fl features distinct and unmatched advantages for SUMO detection in biological specimen: i) unlike known SUMO-binding antibodies, KmUTAG-fl fusion proteins are small (˜50 kDa), monomeric protein fragments that can be produced recombinantly in large quantities; ii) KmUTAG-fl fusion proteins reliably trap and label SUMO-modified proteins with high affinity (e.g. human SUMO1 (KD) of 12.9 nM); iii) using a simply staining protocol, KmUTAG-fl can localize and label SUMO-modified proteins in fixed cells; iv) the specificity of KmUTAG-fl is high for SUMO-modified proteins but low for unconjugated SUMO, resulting in reduced background signals; v) KmUTAG-fl can reliably detect a DNA-damage-dependent relocalization of SUMO-modified proteins in mammalian prostate cancer cells in response to UV and oxidative stress (hydrogen-peroxide).

Further, it has been shown that during unperturbed cell cycle progression, cells maintain balanced levels of SUMO conjugates. In contrast, eukaryotic cells that are exposed to proteotoxic and/or genotoxic insults mount a cytoprotective SUMO-Stress Response (SSR). One hallmark of the SSR is a rapid and massive increase of SUMO conjugates in response to oxidative, thermal, and osmotic stress. The inventors used a recombinant fluorescent SUMO biosensor, KmUTAG-fl, to investigate differences in the SSR in a normal human prostate epithelial cell line immortalized with SV40 (PNT2) and two human prostate cancer cell lines that differ in aggressiveness and response to androgen (LNCaP and PC3). In cells that grow unperturbed, SUMO is enriched in the nuclei of all three cell lines. However, upon 30 minutes of exposure to ultraviolet radiation (UV) and oxidative stress, significant cytosolic enrichment of SUMO, as measured by KmUTAG-fl staining, was detected. This rapid enrichment in cytosolic SUMO levels was on average 5-fold higher in the LNCaP and PC3 prostate cancer cell lines compared to normal immortalized PNT2 cells. Additionally, this enhanced enrichment of cytosolic SUMO was reversible as cells recovered from stress exposure. The results validate the use of a pan-SUMO trapping protein biosensor, for example KmUTAG-fl, for detecting differences of SUMO levels and localization between normal and cancer cells and provides new evidence that cancer cells may exhibit an enhanced SSR.

Accordingly, the proteins including the fusion proteins, described herein can be used for subcellular localization of one or more SUMO-modified proteins in cells. Moreover, the proteins described herein can detect diseased cells for diagnosing and monitoring diseases and monitoring the treatment of diseases. The present disclosure describes methods of using the proteins and fusion proteins described herein, for visualizing and/or localizing SUMO-modified proteins in different types of cells. The pan-Sumo trapping proteins can be labeled for detecting SUMO-modified proteins in cells in vitro and in vivo.

As examples, the pan-Sumo trapping protein can be labeled with biotin, an enzyme, a fluorescent label, a chemiluminescent label, a radioactive label, or a colorimetric label. Examples of enzyme labels include horse radish peroxidase (HRP), alkaline phosphatase (AP), glucose oxidase, and beta-galactosidase. Examples of fluorescent labels include organic dyes such as Alexa dyes, FITC, TRITC, and Dylight fluors, and biological fluorophores such as green fluorescent protein (GFP) and R-phycoerythrin. Methods to label proteins are well-known and routinely practiced. The fusion protein can include a fluorescent protein, as described herein, for localizing SUMO-modified proteins in cells. The pan-Sumo trapping proteins and the fusion proteins described herein can also be formulated as nano-gold particles for use as probes for identifying SUMO-modified proteins.

Examples of the different types of cells include mammalian cells, yeast cells, and nematode oocytes. The cells include in vitro cells, such as tissue culture cells or cells from a biological sample, or in vivo cells, such as in a mammal. The cells can be diseased cells or normal (healthy) cells. The diseased cells include cells under genotoxic and/or proteotoxic stress. The term “genotoxic stress” refers to injuries that cause DNA damage and if left unrepaired results in a mutation that blocks DNA replication. The term “proteotoxic stress” refers to stresses that results in impairment of cellular proteins. Genotoxic stress and/or proteotoxic can cause diseases such as cancer, neurodegenerative diseases, and inflammatory diseases. Moreover, diseases, for example infections, can induce genotoxic and/or proteotoxic stress on the cells.

The diseased cells include cancer cells. Examples of cancer cells include cells from prostate cancer, breast cancer, cervical cancer, ovarian cancer, lung cancer, ovarian cancer, pancreatic cancer, colorectal cancer, bladder cancer, lymphoma, skin cancer, stomach cancer, liver cancer, leukemia (blood cancer), and solid tumor cancers. Examples of solid tumor cancers include retinoblastoma, osteosarcoma, neuroblasoma, and soft tissue sarcoma. In embodiments, the proteins described herein can be used to diagnose cancer including, for example, prostate cancer, breast cancer, and cervical cancer.

The diseased cells also include cells under oxidative stress including being inflicted with oxidizing agents, heat shock stress, UV stress, and osmotic stress. Oxidative stress has been shown to cause neurodegenerative diseases such as Alzheimer's disease, Parkinson's disease, amyotrophic lateral sclerosis, and other neurodegenerative disease. Oxidative stress can also cause inflammatory diseases such as arthritis, asthma, Crohn's disease, irritable bowel syndrome, ulcerative colitis, cardiovascular diseases, and autoimmune diseases. Examples of cardiovascular diseases include atherosclerosis, heart failure, heart attack, stroke, cholesterol and plaque formation, and high blood pressure. Examples of autoimmune diseases include diabetes and lupus. In embodiments, the proteins described herein can be used to diagnose diseases associated with oxidative stress.

The diseased cells can also include cells from an infection. Examples of infections include bacterial or viral infections. Examples of bacterial infections include listeriosis anthrax, pneumococcal pneumonia, tuberculosis, tetanus, and typhoid. Examples of viral infections include common cold, influenza, chickenpox, herpes simplex virus type 1 (HSV1), and human immunodeficiency virus (HIV).

Moreover, sumoylation is a reversible process, which has been confirmed by the results obtained by the inventors using prostate cells. When stress is relieved, SUMO-modified proteins in the cytosol decreases (see Example 4). Accordingly, the proteins described herein can be used to monitor the progression of diseases and the treatment of diseases based on the increase and/or decrease of SUMO-modified proteins in a sample of cells. As an example, the detection of a decrease of SUMO-modified proteins in a sample of cells from a subject diagnosed to have a disease can indicate the alleviation of a disease or a treatment is alleviating a disease if a patient is undergoing a treatment.

The cells for detection, diagnosis, or monitoring can be from a biological sample. The term “biological sample” includes any biological sample from a subject, in particular a mammalian subject, typically a human being. The biological sample can be a biological fluid sample or a tissue sample. Examples of biological fluids include urine, blood, blood serum, plasma, bile, fecal aspirate, intestinal aspirate, cerebrospinal fluid, and saliva. The tissue sample can be from swabs (cheek swab) or a biopsy.

The present disclosure additionally describes methods for detecting and identifying SUMO biomarkers in lysates of cultured cells using the proteins described herein.

In embodiments, the proteins described herein, for example, KmUTAG-fl fusion proteins, can be used for the diagnosis of SUMO biomarkers in, for example, prostate biopsies, prostate fluids, cheek swabs, tissue biopsies, and bodily fluids.

The present disclosure additionally describes methods for introducing the proteins described herein, such as KmUTAG-fl fusion protein, into in vitro and in vivo cells for the detection of one or more SUMO biomarkers for different diseases. As an example, the detection of the biomarkers indicates the subject has the disease or can develop the disease. The present disclosure also describes the use of the proteins described herein, such as KmUTAG-fl fusion protein, for the clinical diagnosis of cancer and other diseases associated with stressed cells found in biopsies, fluids, cheek swabs, tissue biopsies, or bodily fluids. The diagnosis can be based on the detection and/or quantitation of SUMO-modified proteins or on biomarkers. In embodiments, the cancer is prostate cancer.

In embodiments, the methods described herein for detection of one or more biomarkers and for detection, diagnosis, and/or monitoring a disease include obtaining a biological sample, contacting or combining the biological sample with a protein described herein, and detecting SUMO-modified proteins. Contacting or combining the biological sample include incubating the protein described herein with the biological sample for a period of time to enable the protein to bind to the SUMO-modified protein. The method can further include quantitating amount of SUMO-modified protein. The method can further include comparing the detected or quantitated amount of SUMO-modified protein with a control sample. As an example, the control sample can be from a healthy subject or from the subject prior to treatment or at an earlier time in the treatment.

The methods described herein can further include affinity purification of the detected SUMO-modified protein and identifying the protein. The identified protein can be a biomarker for a disease.

The present disclosure also describes kits and systems including the proteins described herein. The kits can be used for detecting biomarkers and/or detecting, diagnosing, or monitoring a disease. The kits include a protein described herein, a labeling agent, and a means for detecting, diagnosing, or monitoring a disease. The kits can also include a protein described herein including a label, such as a fusion protein comprising a pan-Sumo trapping protein and a fluorescent protein or fluorescent label, and a means for detecting, diagnosing, or monitoring a disease. The means for detecting, diagnosing, or monitoring a disease can include an apparatus or a system including various components or modules.

Methods disclosed herein include diagnosing and monitoring diseases in subjects (humans, veterinary animals (dogs, cats, reptiles, birds, etc.), livestock (horses, cattle, goats, pigs, chickens, etc.), and research animals (monkeys, rats, mice, fish, etc.). Subjects in need of diagnosis or monitoring of disease or treatment (in need thereof) are subjects having disease or disorders, such as cancer and other diseases associated with oxidative stress.

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, lack of a material effect is evidenced by lack of a statistically-significant reduction in the embodiment's ability to detect stressed cancer cells in vitro or in vivo.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; ±1% of the stated value; or ±any percentage between 1% and 20% of the stated value.

Recitation of ranges of values herein is merely intended to serve as a shorthand method of referring individually to each separate value falling within the range. Unless otherwise indicated herein, each individual value is incorporated into the specification as if it were individually recited herein. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.5, 2.7, 3, 4, 5, 5.1, 5.3, 5.8 and 6. This applies regardless of the breadth of the range. Moreover, any ranges cited herein are inclusive.

All methods described herein can be performed in any suitable order unless otherwise indicated herein or otherwise clearly contradicted by context.

The use of any and all examples, or exemplary language (e.g., “such as”) provided herein is intended merely to better illuminate the claimed subject matter and does not pose a limitation on the scope of claimed subject matter. No language in the specification should be construed as indicating any non-claimed element essential to the practice of the claimed subject matter.

Groupings of alternative elements or embodiments of the claimed subject matter disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Exemplary Embodiments

The following are exemplary embodiments:
1. A pan-Sumo trapping protein, wherein the protein is stress-tolerant and binds SUMO-modified proteins with high avidity.
2. The protein of embodiment 1, wherein the protein is stress-tolerant as compared to ScUlp or a corresponding WT UD or WT Ulp.
3. The protein of embodiment 1 or 2, wherein the protein includes a binding domain for binding SUMO-modified protein, the binding domain comprising an amino acid sequence comprising at least 65%, 70%, 75%, 80%, 82%, 85%, 86%, 87%, 88%, 89%, 90%, 91%, 92%, 93%, 94%, 95%, 96%, 97%, 98%, 99%, or 100% sequence identity with the amino acid sequence of the UD domain of KmUTAG or SEQ ID NO: 2.
4. The protein of any one of embodiments 1-3, wherein the protein includes an amino acid sequence as set forth in SEQ ID NO: 2.
5. A fusion protein, wherein the fusion protein includes the protein of any one of embodiments 1-4 and at least one other protein.
6. The fusion protein of embodiment 5, wherein the at least one other protein includes a fluorescent protein and/or a purification tag.
7. The fusion protein of embodiment 5 or 6, wherein the at least one other protein is a fluorescent (fl) protein selected from the group consisting of mCherry, mPlum, mRaspberry, HcRed-Tandem, mRFP1, mApple, mRuby, mStrawberry, mTangerine, DsRed-Monomer, TagRFP-T, mOrange, dTomatoTandem, Kusabira Orange, mBanana, TagYFP, TagCFP, mCitrine, mECFP, mTagBFP, and mWasabi.
8. The fusion protein of any one of embodiments 5-7, wherein the fl protein is mCherry.
9. The fusion protein of any one of embodiments 5-8, wherein the fusion protein comprises a protein tag for purification.
10. The fusion protein of any one of embodiments 5-9, wherein the protein tag is an affinity tag.
11. The fusion protein of any one of embodiments 5-10, wherein the affinity tag is a SPOT tag or a His tag.
12. The fusion protein of any one of embodiments 5-11, wherein the fusion protein comprises the amino acid sequence as set forth in SEQ ID NO: 4.
13. The protein of any one of embodiments 1-12, wherein the protein is obtained by recombinant means.
14. A nucleic acid encoding the protein of any one of embodiments 1-13.
15. The nucleic acid of embodiment 14, wherein the nucleic acid includes a sequence comprising at least 65%, 70%, 75%, 80%, 82%, 85%, 86%, 87%, 88%, 89%, 90%, 91%, 92%, 93%, 94%, 95%, 96%, 97%, 98%, 99%, or 100% sequence identity with the nucleic acid sequence encoding the UD domain of KmUTAG or SEQ ID NO: 1.
16. A vector comprising the nucleic acid of embodiment 14 or 15.
17. The vector of embodiment 15, wherein the vector is an expression vector.
18. A host cell including the nucleic acid of embodiment 14 or 15 or the vector of embodiment 16 or 17.
19. A method of preparing a protein of any one of embodiments 1-13, wherein the method includes introducing the vector of embodiment 16 or 17 into a host cell and culturing the host cell under conditions that allow the expression of the protein.
20. A method of preparing a protein of any one of embodiments 1-13, wherein the method includes inserting the nucleic acid of embodiment 14 or 15 into a vector, introducing the vector into a host cell, and culturing the host cell under conditions that allow the expression of the protein.
21. A method of preparing a protein of any one of embodiments 1-13, wherein the method includes culturing the host cell of embodiment 18 under conditions that allow the expression of the protein.
22. The method of embodiment 19-21, wherein the method further includes recovering and/or storing the protein.
23. The method of embodiment 22, wherein recovering the protein includes purifying the protein by affinity purification.
24. The method of embodiment of 22 or 23, wherein the protein is stored cryogenically.
25. A method of detecting one or more SUMO-modified proteins in a biological sample, wherein the method includes combining the biological sample with a protein of any one of embodiments 1-13 and detecting one or more SUMO-modified proteins.
26. The method of embodiment 25, wherein the biological sample includes an in vitro sample of cells or wherein the biological sample includes in vivo cells.
27. A method of diagnosing a disease in a subject, wherein the method includes combining a biological sample from a subject with the protein of any one of embodiments 1-13 and detecting one or more SUMO-modified proteins.
28. A method of monitoring a disease in a subject, wherein the method includes combining a biological sample from a subject with the protein of any one of embodiments 1-13 and detecting one or more SUMO-modified proteins.
29. The method of any one of embodiments 25-28, wherein the method further comprises labeling the protein of any one of claim 1-6 or 9-11 prior to combining the biological sample with a protein.
30. The method of embodiment 29, wherein the protein is labeled with biotin, an enzyme, a fluorescent label, a chemiluminescent label, a radioactive label, or a colorimetric label.
31. The method of any one of embodiments 25-29, wherein the method of detecting one or more SUMO-modified includes quantitating the one or more SUMO-modified modified protein.
32. The method of any one of the embodiments 25-31, wherein the method further includes comparing the results of the detecting with a control.
33. The method of embodiment 32, wherein the control includes the biological sample from a healthy subject or from the subject of an earlier time of the disease or in a treatment therapy.
34. The method of any one of embodiments 25-33, wherein the detected one or more SUMO-modified proteins are biomarkers of a disease associated with genotoxic and/or proteotoxic stress.
35. The method of any one of embodiment 25-34, wherein the biological sample is from a mammalian subject and the sample is a biological fluid sample or tissue sample.
36. The method of embodiment 35, wherein the biological sample is obtained from urine, blood, blood serum, plasma, bile, fecal aspirate, intestinal aspirate, cerebrospinal fluid, saliva, swab (e.g. from cheek), or a biopsy.
37. The method of any one of embodiments 25-36, wherein the biological sample is from a subject diagnosed with a disease associated with genotoxic and/or proteotoxic stress.
38. The method of any one of embodiments 25-37, wherein the method detects, diagnoses, or monitors cancer, a neurodegenerative disease, an inflammatory disease, or an infection.
39. The method of any one of embodiments 25-38, wherein the method detects, diagnoses or monitors prostate cancer, breast cancer, cervical cancer, solid tumors, and leukemia.
40. The method of any one of embodiments 25-38, wherein the method detects, diagnoses, or monitors Alzheimer's disease, Parkinson's disease, or amyotrophic lateral sclerosis.
41. The method of any one of embodiments 25-38, wherein the method detects, diagnoses, or monitors prostate cancer, breast cancer, or cervical cancer.
42. The method of any one of embodiments 25-38, wherein the method detects, diagnoses, or monitors arthritis, asthma, Crohn's disease, irritable bowel syndrome, ulcerative colitis, cardiovascular diseases, or autoimmune diseases.
43. The method of any one of embodiments 25-38, wherein the method detects, diagnoses, or monitors bacterial or viral infections including listeriosis and herpes simplex virus type 1 (HSV1).
44. The method of any one of embodiments 25-42, wherein the method detects, diagnose, or monitors the alleviation of the disease or treatment of the disease.

### EXAMPLES

Introduction. SUMO is an essential and highly conserved, small ubiquitin-like modifier protein. In this protocol we are describing the use of a stress-tolerant recombinant SUMO-trapping protein (kmUTAG) to visualize native, untagged SUMO conjugates and their localization in a variety of cell types. The inventors provide a novel method to study the sumoylation of proteins and their sub-cellular localization in mammalian cells and nematode oocytes. The method utilizes a recombinant SUMO-modified-trapping protein fragment, KmUTAG, derived from the Ulp1 SUMO protease of the stress-tolerant budding yeast Kluyveromyces marxianus. The properties of the KmUTAG have been adapted for the purpose of studying sumoylation in a variety of model systems without the use of antibodies. KmUTAG has several advantages in comparison to antibody-based approaches for the study of SUMO. This stress-tolerant SUMO-trapping reagent is produced recombinantly, it recognizes native SUMO isoforms from many species, and unlike commercially available antibodies it shows reduced affinity for free, unconjugated SUMO. The results confirm the localization of SUMO conjugates in mammalian tissue culture cells and nematode oocytes.

### Example 1. Generation of Bacterial Expression Plasmid to Produce Recombinant KmUTAG-Flmc

A codon-optimized KmUTAG ORF was synthesized by Genewiz Inc. and inserted in the mammalian expression vector pmCherry-C1 (Clontech.com) to form mCherry-KmUTAG plasmid BOK1399 (Peek et al., 2018).

To generate a bacterial over-expression clone the mCherry-KmUTAG ORF was PCR-amplified with EcoR1 and HindIII overhangs from BOK1399 using primers 199736451 (EcoR1mcherryFWD) and 199736452 (KmULP1(mam_opt)STOP_HINDIII). The resulting PCR product was PCR-cloned into a Strataclone PCR cloning vector (agilent.com). The cloned mCherry-KmUTAG fragment was recovered after EcoR1/HindIII double digest and ligated into the EcoR1/HindIII digested SPOT-tag plasmid pEV1 (chromotek.com).

For expression of recombinant SPOT-tagged KmUTAG-flmc, the resulting plasmid was transformed into BL21-STAR(DE3) cells (Muench et al., 2003). For over-expression, 75 ml of bacterial log-phase SOC cultures containing 1% glycerol were grown at 18° C. for 20 hours. Visibly “pink” cell pellets were recovered by centrifugation, resuspended in 1 ml SPB+5 mM TCEP+1 mM AEBSF (Peek et al., 2018), washed once, and then sonicated 3×10% duty cycle for 20 sec each. Lysates were clarified and pink supernatant was [[was]] incubated with 20 ul magnetic SPOT-trap beads for 1.5 hr. Beads were eluted into 100 ul dilution buffer (Chromotek Inc) containing 1.4 mM SPOT peptide (no TCEP) for 1.5 h. The “pink” supernatant containing recombinant SPOT-tagged KmUTAG-mCherry (KmUTAG-flmc) was analyzed and quantitated using SDS-PAGE gels and 20% glycerol [final] was added to the recombinant KmUTAG-flmc before snap freezing in liquid nitrogen and long-term storage at −80° C.

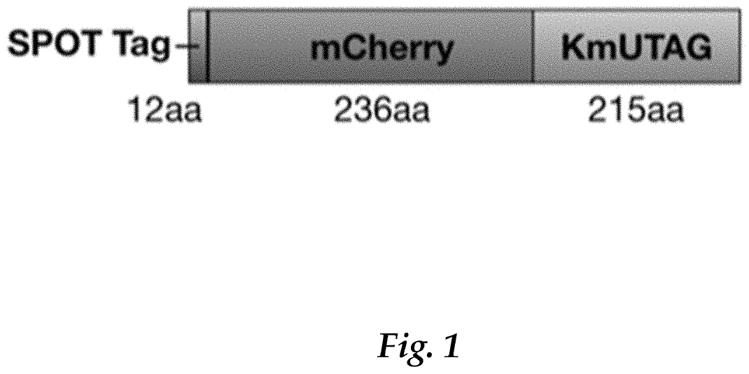

In summary, KmUTAG-fl is a recombinant, mCherry-tagged SUMO-trapping protein. To produce kmUTAG-fl, a codon-optimized mCherry-kmUTAG was cloned into the pSPOT1 bacterial overexpression plasmid (FIG. 1) (ChromoTek®). After induction, the kmUTAG-fl protein was purified on Spot-TRAP, eluted, and frozen until further use. To ensure the SUMO-trapping activity of KmUTAG-fl, the binding to SUMO1-conjugated beads and precipitation of a SUMO-CAT fusion protein was confirmed.

### Example 2: SUMO Detection in Fixed Tissue Culture Cells Using Recombinant KmUTAG-flmc SUMO-Trapping Protein

Experimental Protocol:

2.1 Tissue culture cells of choice were grown on 22 mm round cover slips in 6-well TC plates until 70-80% confluent. All subsequent steps were performed in the 6-well plate.

2.2 The cells were washed briefly with 1 ml dPBS.

2.3 Fixation: The cells were fixed with 4% Paraformaldehyde (PF) for 20 min at room temperature. Two milliters of dPBS/well containing 4% paraformaldehyde were used. All steps using PF were performed in a laboratory safety hood and PF must be disposed of properly.

2.4 The fixed cells were washed 3 times in 1 ml dPBS while nutating, 5 min for each wash.

2.5 Permeabilization: the cells incubated for 15 min with 0.1% Triton X-100 in dPBS

2.6 The cells were washed 3 times in 1 ml dPBS while nutating plate, 5 min for each wash.

2.7 The cells were incubated with 500 ul 0.1M Glycine-HCL (pH 2.0) for 10 seconds, and the pH was neutralized immediately with 500 ul of 10×SUMO Protease Buffer (SPB).

2.8 The cells were washed 3 times in 1 ml dPBS while nutating plate, 5 min for each wash.

2.9 The coverslips were removed from the well and placed in a humidity chamber. Incubations on the coverslip proceeded on the coverslip as follows:
2.9.1 KmUTAG-fl only: 1 ug KmUTAG-fl was mixed in a tube with 1×SPB containing 5 mM TCEP. The mix was pipetted onto the cells on the coverslip and incubated at room temperature for 1 hr in the humidity chamber.
2.9.2 KmUTAG-fl and anti-SUMO1 antibody co-staining:
i) 1 ug KmUTAG-fl and 0.5 ul SUMO2/3 8A2 (obtained for Developmental Studies Hybridoma Bank (X.-D. Zhang et al., 2008)) were mixed in a tube with 100 ul blocking buffer. The mix was pipetted onto the coverslip and incubated in room temperature for 1 hr.
ii) The cells on the coverslip were washed 3 times with 200 ul dPBS, 5 min for each wash.
iii) 0.5 ul anti-mouse Alexa Fluor 488 conjugated antibody was mixed in a tube with 100 ul Blocking buffer, pipette the mix onto the coverslip, incubate in room temperature for 1 hr.

2.10 200 μl dPBS was pipetted on each coverslip and left in place for 10 min to wash the coverslips. The wash was repeated 2 more times.

2.11 After removing the last wash, the coverslip was inverted onto a pre-cleaned microscopy slide with a drop of FLUORO-GEL 11 with DAPI and stored in −20° C. freezer overnight before viewing under the microscope. Filters for DAPI and Texas red were used for visualization.

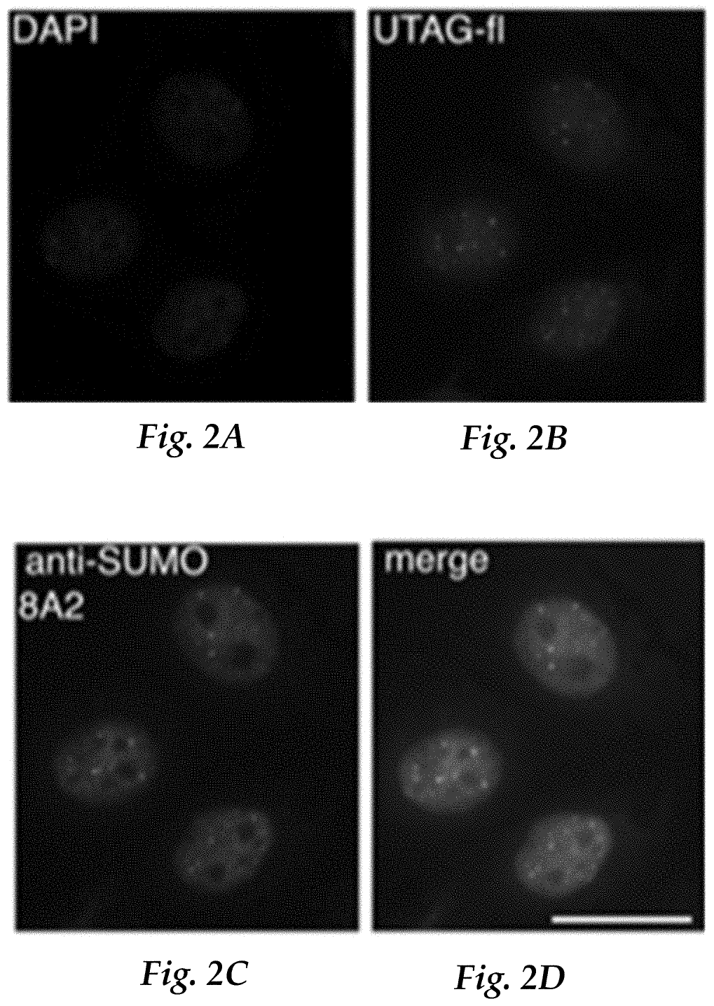

Results. KmUTAG-flmc incubated with fixed PNT2 cells showed a distinct nuclear staining when observed using the appropriate filter set (Chroma) and a 100× oil-immersion objective on an Epifluorescent Zeiss Axioplan Microscope (FIG. 2B). Both diffuse nuclear staining and distinct nuclear foci were visible. Nuclear localization was confirmed using co-staining with DAPI (FIG. 2A). Consistent with the SUMO-trapping activity of KmUTAG-flmc, the nuclear localization pattern was reminiscent of SUMO2/3 staining. Co-staining with anti-SUMO2/3 8A2 antibody (FIG. 2C) confirmed the co-localization of kmUTAG-fl with the SUMO2/3 signal (FIG. 2B). This validates the efficacy of KmUTAG-flmc to detect SUMO2/3 in mammalian cells.

### Example 3: SUMO Detection in Fixed Nematode Gonads Using KmUTAG-Fl

Experimental Protocol:

3.1 Adult hermaphrodites were transferred to an 8 microliters droplet of egg buffer (Edgar, 1995) on a plus-charged slide that had been additionally subbed with poly-L-lysine. Gonads were released from the worms using 27.5 gauge needles. Proceeded with either antibody labeling or KmUTAG-fl labeling.

3.2. Antibody labeling:
3.2.1 Fixation: Samples were freeze-cracked in liquid nitrogen and fixed overnight in −20° C. methanol.
3.2.2 Blocking: The fixed samples were washed 3 times in 1×PBS, and then blocked for 20 minutes in PBS containing 0.5% BSA and 0.1% Tween 20.
3.2.3 Primary antibody incubation: The samples were incubated overnight with anti-SUMO 6F2 antibody (1:10) at 4° C. (obtained for Developmental Studies Hybridoma Bank (Pelisch et al., 2014))
3.2.4 Secondary antibody incubation: The samples were then washed for 2 minutes in 1×PBS and incubated with Dylight 488 goat-antimouse antibody (1:200) for 1.5 hours at room temperature.
3.2.5 Mounting: The samples were then washed for 2 minutes in 1×PBS, dip in dH20, and mounted on slides with Fluoro Gel with DABCO and DAPI.

3.3 KmUTAG-fl labeling:
3.3.1 Fixation: Equal volume of 8% paraformaldehyde was added to the samples for a final concentration of 4%. The samples were fixed for 10 minutes and then quenched in 1×PBS containing 0.1 M glycine for at least 5 minutes.
3.3.2 Permeabilization: The samples were then washed for 5 minutes in 1×PBS and permeabilized in 1×PBS containing 0.1% Triton-X for 10 minutes.
3.3.3 Glycine-HCl treatment: The samples were then washed for 5 minutes in 1×PBS. 200 ul of 0.1M Glycine-HCl (pH=2.0) was added to the samples for 10 seconds. 200 ul of 10×SPB was added immediately to neutralize the pH.
3.3.4 The samples on the slides were returned to the coplin jar and washed for 5 mins in 1×PBS.
3.3.5 KmUTAG-fl incubation: The slides were removed from the wash. 100 ul 1×SPB+5 mM TCEP containing 2 ug of KmUTAG-fl was pipetted onto the nematodes on the slides. The slides were incubated in the humidity chamber for 1 hr, without rocking.
3.3.6 The slides were returned to the coplin jar and washed for 15 mins in 1×PBS
3.3.7 Mounting: The slides were removed from the wash, and a Kimwipe was used to dry around the sample of each slide. The slides were mounted with 5 ul Fluoro Gel with DABCO and DAPI and stored at 4° for viewing the next day.

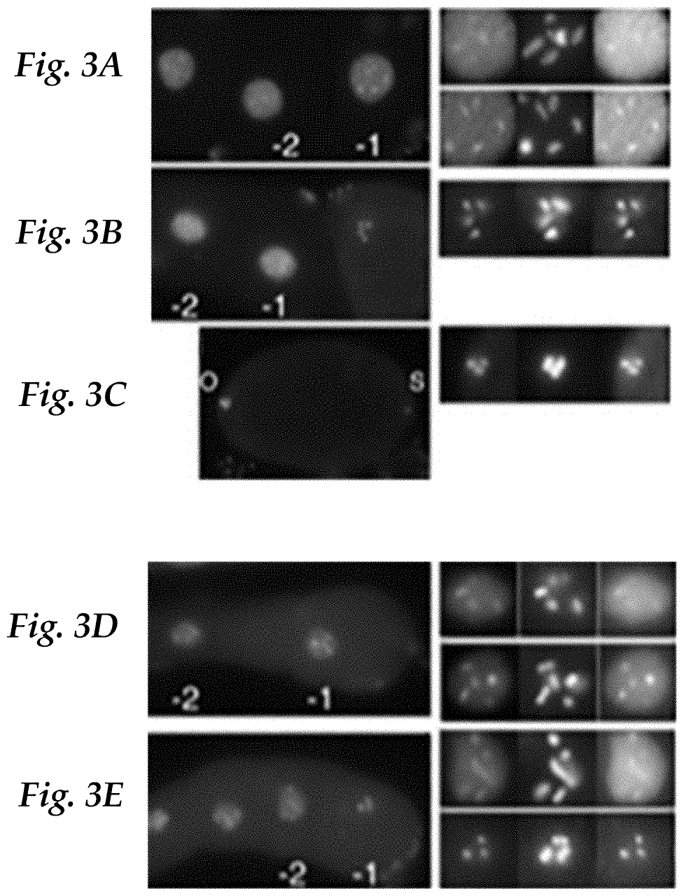

Results. Hermaphrodites were dissected and gonads were stained with either anti-SUMO antibody as a control or KmUTAG-flmc. In both preparations, developing oocytes revealed previously reported SUMO patterns (Pelisch et al., 2017). Developing oocytes exhibited diffuse nuclear staining before the antibody or KmUTAG-flmc begun to label the chromosomes (FIG. 3). By nuclear envelope breakdown entry into metaphase of meiosis 1, both the antibody (FIGS. 3B and 3C) and the KmUTAG signal (FIG. 3E) coalesced in the mid-section (the ring complex) between mid-bivalent chromosomes co-stained with DAPI. These results validate KmUTAG-flmc for the study of meiosis and SUMO-related processes in C. elegans and possibly other nematodes.

### Example 4. Detection of Rapidly Accumulating, Stress-Induced SUMO in Prostate Cancer Cells by a Fluorescent SUMO Biosensor

Introduction. An early study by Saitoh and Hinchey showed that exposure to proteotoxic and genotoxic stressors led to an extremely rapid increase of SUMO-conjugated proteins in the cell, often within minutes (Saitoh & Hinchey, 2000). For example, SUMO-modification is rapidly enhanced when cells are subjected to reagents and conditions that damage proteins (e.g. hydrogen peroxide and increased temperature) or DNA (e.g. UV irradiation). This phenomenon is now termed the SUMO stress response or SSR (Lewicki, Srikumar, Johnson, & Raught, 2015). A long list of sumoylated proteins that accumulate in response to stress, including many chromatin remodeling factors and transcription factors, have been identified using proteomic approaches (reviewed in (Golebiowski et al., 2009 and references therein). It remains unclear however, why this massive SUMO modification event unfolds as cells experience stress.

There is good evidence that organisms as diverse as yeast and humans utilize SUMO modification in their cellular stress response. This is borne out by the reduced stress tolerance of cells that lack intact sumoylation pathways. In yeast, a number of non-essential genes involved in the response to proteotoxic and genotoxic stress result in lethality when paired with genetic defects in sumoylation and desumoylation (reviewed in Seeler & Dejean, 2017). Correspondingly, mammalian cells that are depleted of SUMO or the SUMO protease SENP1 show reduced ability to survive acute heat shock or exposure to ionizing radiation (Golebiowski et al., 2009; R.-T. Wang, Zhi, Zhang, & Zhang, 2013b). This suggests that sumoylation plays an important role in the response to proteotoxic and genotoxic stress.

SUMO's role in stress tolerance, however, remains enigmatic at best. Findings from three recent studies arrived at different, yet non-mutually exclusive conclusions (Golebiowski et al., 2009; Lewicki et al., 2015; Liebelt et al., 2019). One study found that, in mammalian cells, SUMO isoforms served a chaperone-like function and maintained the homeostasis of large chromatin-associated nuclear proteins during stress (Seifert, Schofield, Barton, & Hay, 2015). Another found that sumoylation temporarily stabilized denatured proteins after heat shock, preventing them from aggregating before proteasome-mediated degradation (Liebelt et al., 2019). A third study found that environmental stress induced a wave of transcription-coupled sumoylation and SSR was found to be blocked when transcription was inhibited (Lewicki et al., 2015).

A rapid increase in sumoylation and SSR occurs due to increased activity of SUMO E3 ligases, possibly coupled with a decrease in SUMO protease activity. This is borne out by the finding that initiation of the SSR in stressed cells is caused primarily by the E3 SUMO ligase Siz1 in yeast, and the combined effort of the orthologous SUMO ligases PIAS1-4 in heat-stressed osteosarcoma cells (Lewicki et al., 2015; Seifert et al., 2015). Additionally, several SUMO proteases (Ulp1 in yeast and SENP 1, 2, 3, 7 in mammalian cells) are inactivated due to heat and/or oxidative stress, suggesting they may act as stress sensors (Pinto et al., 2012). Inactivation of these SUMO proteases invariably results in an accumulation of SUMO-conjugated proteins as well as SUMO chains. Recent research by the inventors indicates that the S. cerevisiae SUMO protease Ulp1 is unable to bind SUMO in the presence of extremely low levels of hydrogen peroxide [0.006%], underscoring a potential role of SUMO proteases as redox sensors (Peek et al., 2018). One notable exception, the mammalian SUMO protease SENP6 is not inactivated by heat stress and becomes recruited to chromatin (Pinto et al., 2012; Seifert et al., 2015). SENP6 is similar to the yeast SUMO protease Ulp2, which in turn is required for recovery from the SSR in yeast.

This suggests that both SENP6 and ULP2 are involved in removing SUMO chains as a necessary step for the recovery of cells from the SSR (Lewicki et al., 2015). Additionally, low levels of oxidative stress also rapidly disable the SUMO E1 (Uba2) and E2 (Ubc9) enzymes—via the formation of a disulfide bond between their catalytic cysteine residues—raising the question of how sumoylation can become amplified dramatically during the SSR when SUMO conjugation is halted (Bossis & Melchior, 2006). Although the answer is unknown, a pre-existing pool of Ubc9 with non-covalently bound SUMO, which has been shown to be involved in the formation of SUMO chains, may hint at the answer (Knipscheer, van Dijk, Olsen, Mann, & Sixma, 2007).

The SSR pathway exists in single cell eukaryotes (e.g. yeasts), in normal mammalian cells, and is generally dysregulated in cancer cells (Seeler & Dejean, 2017). Cancer cells are subjected to a host of adverse conditions including hypoxic environments within tumors, attack by tumor-invading immune cells, and rampant aneuploidies that dysregulate cellular proteostasis. Consequently, cancer cells rely on enhanced stress response pathways to survive under these conditions. In general, sumoylation enzymes, such as activating (E1) and conjugating (E2) enzymes have been found to be elevated in tumors, potentially altering the activity of dozens of SUMO-modified tumor suppressors, oncoproteins, and stress response proteins, including heat-shock proteins (e.g. Hsp90), hypoxia-inducible factors (e.g. Hif1A), and inflammatory signaling factors (e.g. IkBalpha) (reviewed in (Seeler & Dejean, 2017)). Specifically, the overexpression of the SUMO ligase Ubc9 in cancers has been linked to poor treatment outcomes and Ubc9 overexpression may be a useful biomarker for cervical cancer (Mattoscio, 2015; Wu, Zhu, Ding, Beck, & Mo, 2009). However, elevated levels of some SUMO proteases have also been linked to breast and prostate cancer development (Karami et al., 2017; Q. Wang et al., 2013a). These examples suggest that accelerated SUMO dynamics, both sumoylation and desumoylation, are at play in the stress resilience of cancer cells.

To visualize SUMO within cells with the KmUTAG, the inventors developed a Recombinant Fluorescent SUMO Biosensor KmUTAG-fl (Yin, Harvey, Shakes, & Kerscher, 2019). KmUTAG-fl is a recombinant mCherry-tagged SUMO-trapping fusion protein. This stress-tolerant pan-SUMO specific biosensor is produced recombinantly in bacteria. Once purified it recognizes and traps native SUMO-conjugated proteins and SUMO chains in fixed permeabilized cells. This biosensor protein compares favorably to staining protocols with SUMO specific antibodies as it has reduced affinity for free, unconjugated SUMO. Meanwhile, it can be used to analyze SUMO variants from additional model and non-model systems. In the present study, KmUTAG-fl was used to detect SUMO in a variety of human prostate cells lines; a nontumorigenic SV40-immortalized prostate epithelial cell line (PNT2), an aggressive androgen-insensitive prostate cancer cell line derived from bone metastasis with high metastatic potential (PC3), and a hormone-sensitive metastatic prostate cancer cell line derived from lymph node metastasis with low metastatic potential (LNCaP). Using the fluorescent KmUTAG-fl biosensor, SUMO levels in the nuclei and extra-nuclear compartment (cytosol) in untreated, UV-treated, and H202-treated cells were compared. Significant differences were detected in the SUMO profiles between normal and cancer prostate cancer cells. After stress exposure, both prostate cancer cell lines showed a cytosolic SUMO enrichment that was 5-fold higher than normal PNT2 cells. The cytosolic SUMO enrichment was detected within 30 min of stress exposure and was completely reversible after recovery in fresh media. While there was a clear difference between cancer and normal prostate cells, a difference between cells exhibiting low (LNCaP) and high (PC3) metastatic potential was not detected. We posit that differences in the SSR are linked to the enhanced robustness of cancer cells and therefore, SUMO profiles as visualized using the KmUTAG-fl biosensor can be used to differentiate normal and tumorigenic cells.

4.1 Cell Culture and Maintenance

PC3, PNT2, and LNCaP cells were grown in RPMI media with 10% heat inactivated FBS (Thermo Fisher Scientific #10438018) and 1% antifungal/antibiotic (anti/anti) (Thermo Fisher Scientific #15240062). All cells were grown at 37° C. in a humidified incubator which is kept constant at 5% CO₂.

4.2 Expression of kmUTAG-fl and In Vitro Assays

A codon-optimized bacterial overexpression clone of mCherry-KmUTAG was generated as previously described (Example 1, Yin et al., 2019). KmUTAG-fl biosensor was over-expressed in BL21-STAR(DE3) cells (Muench et al., 2003). Purification of KmUTAG-fl in these cells was as previously described (Yin et al., 2019) except that KmUTAG-fl was bound to magnetic SPOT-trap beads (Chromotek). To assess SUMO-trapping activity of KmUTAG-fl, SUMO-binding reactions were performed on SUMO1 beads and with recombinant SUMO-CAT fusion protein (Peek et al., 2018).

For SUMO staining, PC3, PNT2 or LNCaP cell were grown to 80% confluency and counted using a hemocytometer. Each well in the 6-well plate (Fisher Scientific 07-200-83) received 300,000 cells in 2 mL media. Cells were incubated for 24 hours until 80% confluent. After fixing the cells with fresh 4% Paraformaldehyde (PF) for 20 minutes at room temperature, cells were permeabilized for 15 min with 0.1% Triton X-100 in dPBS. Next, the cells were incubated with 0.1M Glycine-HCL (pH 2.0) for 10 seconds. The pH was immediately neutralized with 500 μL of 10×SPB (500 mM Tris-HCL, pH 8.0 2% NP-40, 1.5M NaCl) (Peek et al., 2018) and coverslips were removed from the well and placed into humidity chambers. For KmUTAG-fl staining, 2 ug of recombinant KmUTAG-fl was added to 100 μL 1×SPB containing 5 mM TCEP. The mix was transferred onto the coverslip and incubated at room temperature for 1 hr in the humidity chamber. For KmUTAG-fl and anti-SUMO2/3 antibody co-staining, 2 ug of KmUTAG-fl and 0.5 μL SUMO2/3 8A2 (obtained for Developmental Studies Hybridoma Bank—DSHB Hybridoma Product SUMO-2 8A2 (X.-D. Zhang et al., 2008)) were mixed with 100 μL blocking buffer, pipetted onto the coverslip, and incubated in room temperature for 1 hr. After washing the coverslips with dPBS, 0.5 μL anti-mouse Alexa Fluor 488 conjugated antibody (Jackson ImmunoResearch 115-545-003) in 100 μL Blocking buffer (Prometheus 20-313), was pipetted onto the coverslip of KmUTAG-fl and SUMO2/3 co-staining slides, and incubated at room temperature for 1 hr. After washes with dPBS, the coverslips were inverted onto pre-cleaned microscopy slides with FLUORO-GEL II with DAPI (Electron Microscopy Sciences 50-246-93). The slides were stored at −20° C. overnight before viewing under the microscope.

4.3 Stress Treatment

To test the SUMO Stress Response (SSR) to UV damage, cells were grown until ˜80% confluent on coverslips and subjected to UV irradiation before proceeding with fixation and staining. The coverslips were first transferred from the 6-well plate to a humidity chamber. Excess media on the coverslip was removed and the humidity chamber containing the coverslip was put into a UV chamber (GS Gene Linker). The UV intensity was adjusted per manufacturer's protocol. After irradiation, coverslips were immediately placed back into culture medium and placed back into the tissue culture incubator for an additional 30 min. Fixation and staining was completed as detailed above.

For peroxide stress treatment, H₂O₂ was added from a 3M stock solution to 2 mL cultures in a 6-well plate to achieve the desired final concentration. Cells were then incubated for an additional 30 min in the tissue culture incubator. After incubation, the tissue culture supernatant was removed and the cells were washed in dPBS before fixation and staining with KmUTAG-fl.

4.4 Microscopy and Data Analysis

Images were acquired using a fully automated Nikon A1R inverted confocal microscope or a Zeiss Axioscope using the appropriate filter sets. Fluorescence staining intensity was quantified using CellProfiler (www.cellprofiler.org (McQuin et al., 2018)). Nuclei and cytosol of imaged cells were automatically detected as DAPI-stained nuclei and kmUTAG-fl stained nuclei and cytosol, respectively. For normalization between images, the background fluorescence intensity between cell features was subtracted from the mean intensity of each compartment of individual cells to obtain the cytoplasmic and nuclear staining intensity. The relative cytosolic enrichment was calculated as the ratio between cytoplasmic and nuclear staining intensities per cell. Number of cells used for evaluation is listed in the individual figure legends. Unpaired parametric T-test was used to assess significant differences in staining intensity of cytosolic and nuclear kmUTAG-fl before and after stress exposure. Significant level signs were displayed to indicate the result of the T-test (NS: P>0.05, *P≤0.05, **P≤0.01, ***P≤0.001). The percentage difference between mean fluorescence intensity levels per treatment group was calculated to further describe the change in kmUTAG-fl signal intensity before and after stress. Data was graphed using R software (scripts available upon request).

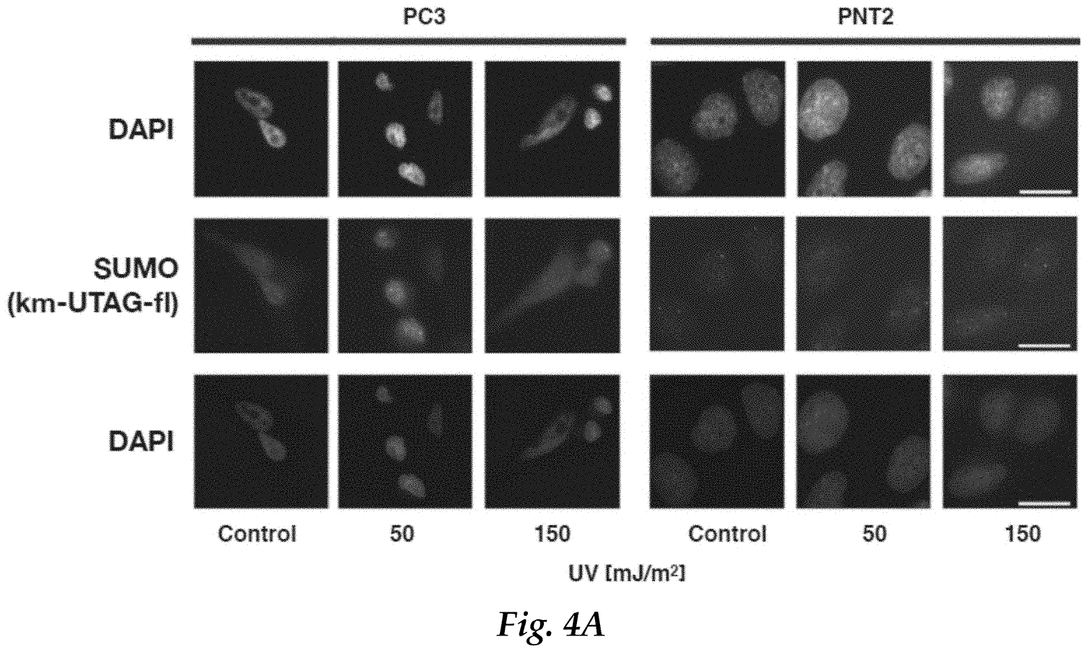

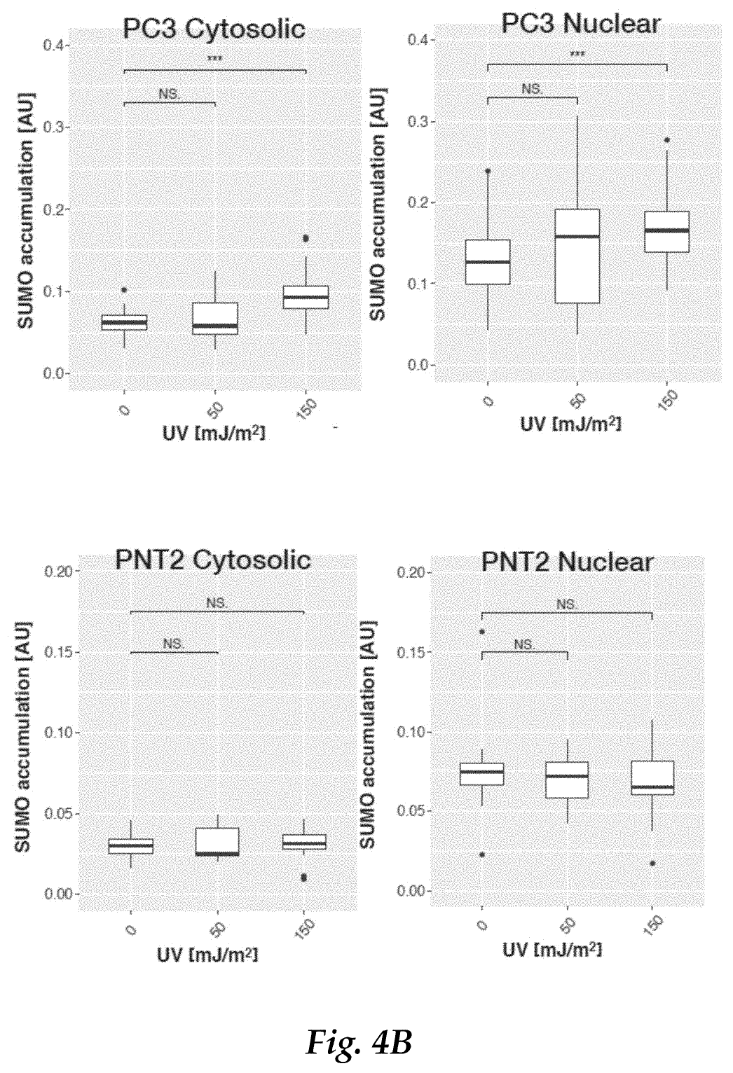

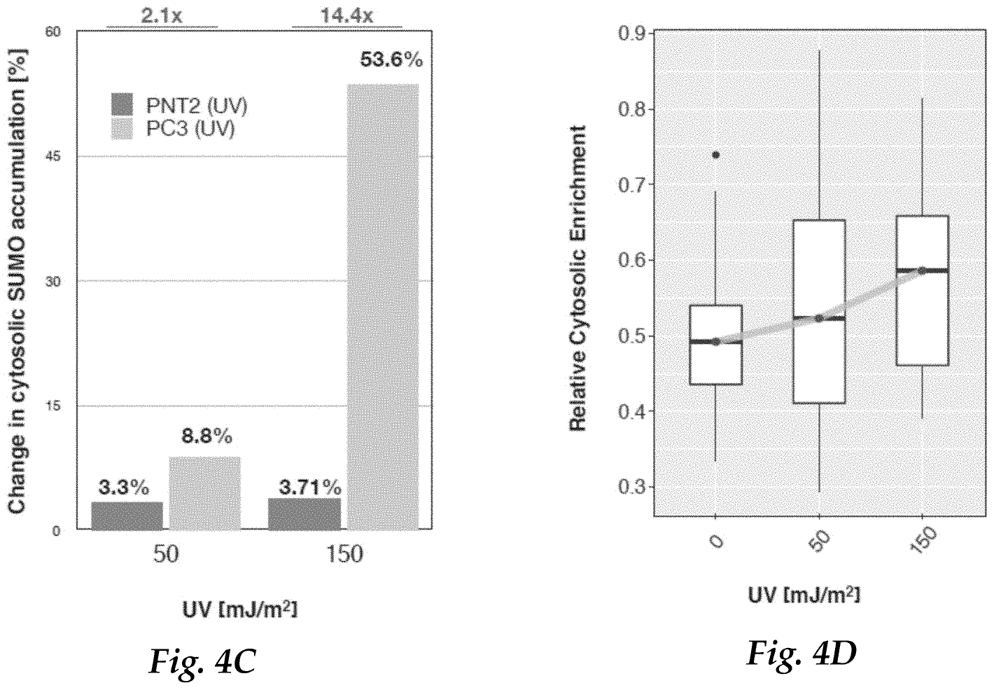

Results. Cytosolic SUMO levels increased in PC3 prostate cancer cells upon UV-irradiation. It was previously shown that KmUTAG-fl is a single-chain recombinant Pan-SUMO binding protein that reliably detected SUMO conjugates in fixed nematode gonads and in mammalian cells (Yin et al., 2019). In unperturbed mammalian cells, the KmUTAG-fl signal co-localized with SUMO2 in the nucleoplasm and in distinct nuclear foci (Peek et al., 2018; Yin et al., 2019). To investigate the differences of the SSR in normal and cancer cells, KmUTAG-fl was used to investigate the impact of UV-induced stress on SUMO levels in two human prostate cell lines, immortalized normal PNT2 cells derived from prostate epithelium and PC3 adenocarcinoma cells with high metastatic potential (Berthon, Cussenot, Hopwood, Leduc, & Maitland, 1995; Tai et al., 2011). PC3 and PNT2 cells were grown on coverslips and subjected to various doses of UV irradiation (50 mJ/m² and 150 mJ/m²). After irradiation, the cells were allowed to recover for 30 min in culture medium before fixing and staining with KmUTAG-fl. A rapid increase in KmUTAG-fl staining after UV exposure (150 mJ/m²) was visually apparent in PC3 cells (FIG. 4A left panel) but was not discernible in PNT2 cells (FIG. 4A right panel). Levels of KmUTAG-fl in PC3 cells were measured and quantified using CellProfiler, revealing a significant increase in SUMO levels between the un-irradiated [zero “0” mJ/m²] and irradiated [150 mJ/m²] samples (FIG. 4B—top panels). Specifically, after UV treatment (150 mJ/m2). It was found KmUTAG-fl staining in the cytosol (denoting the extra-nuclear region of the cell) increased by ˜54% in PC3 cells (FIG. 4C). In comparison, no significant change in SUMO accumulation was detected in the cytosol of PNT2 (FIG. 4B bottom left panel). Therefore, the difference in cytosolic SUMO levels between UV-treated PC3 and PNT2 cells (150 mJ/m2) was approximately 14-fold. Concomitantly, UV-treatment also increased SUMO levels in the nucleus of PC3 cells, albeit only by 27% (FIG. 4B top right panel). No significant change in SUMO accumulation was detected in nuclei of PNT2 (FIG. 4B bottom right panel). Importantly, taking into account nuclear and cytosolic KmUTAG signals, an increasing relative cytosolic enrichment (RCE) of SUMO was recorded after UV irradiation (FIG. 1D). These data indicated that the SSR in PC3 cancer cells involved a significant increase of SUMO or SUMO conjugates in the cytosol.

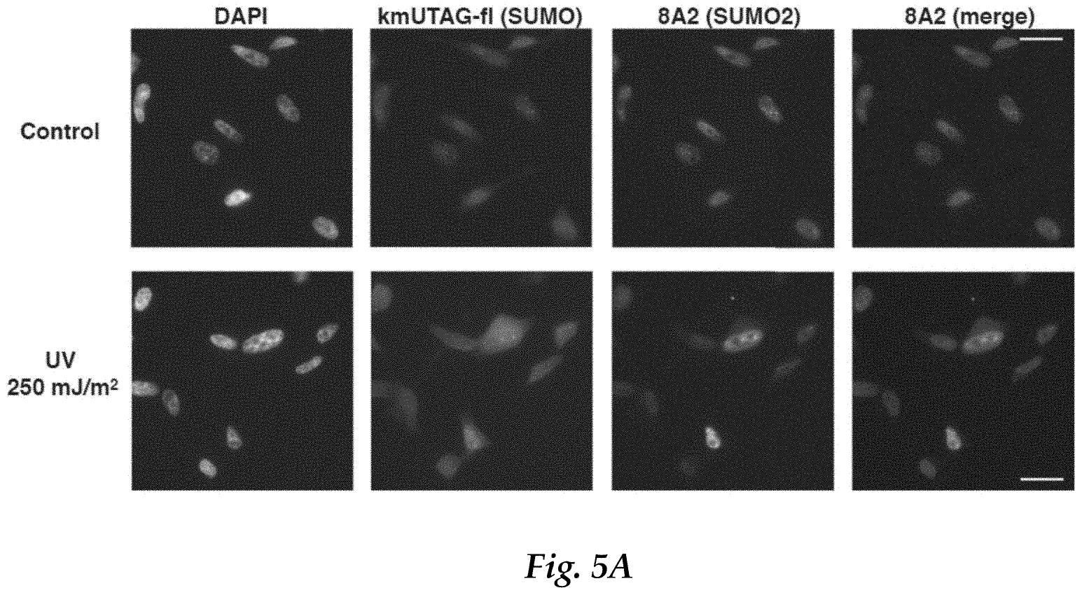

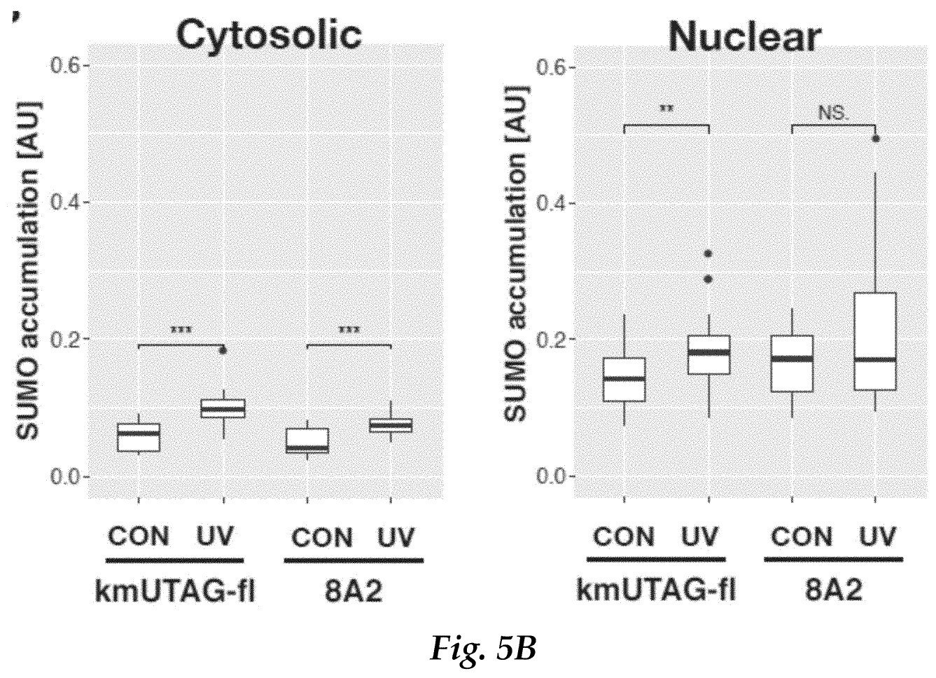

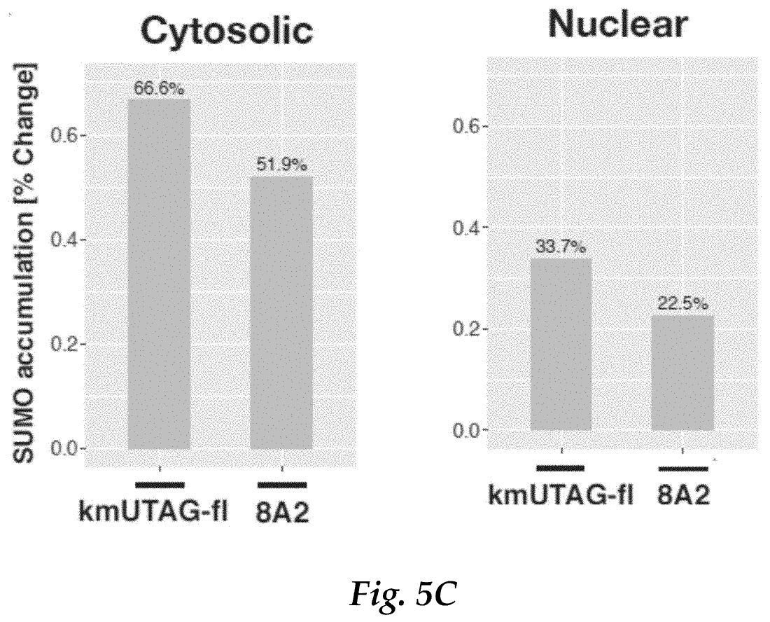

Next, this phenomenon of increased extra-nuclear SUMO accumulation was investigated to determine whether it could be recapitulated using the monoclonal anti-SUMO2 8A2 antibody to visualize SUMO (X.-D. Zhang et al., 2008). Therefore, the ability of KmUTAG-fl and the anti-SUMO2 8A2 antibody to detect a change in the localization and levels of SUMO following UV-irradiation of PC3 cells was compared. As expected, non-irradiated PC3 cells stained with the 8A2 antibody revealed SUMO2 localization in the nucleus. However, upon UV irradiation [250 mJ/m²], SUMO2 was also detected in the cytosol of these cells (FIG. 5A). Quantitation of KmUTAG-fl and 8A2 signal revealed that cytosolic SUMO/SUMO2 levels increased by 67% and 52%, respectively (FIG. 5C). However, unlike the KmUTAG-fl stained sample, 8A2 antibody staining did not reveal a significant change of nuclear SUMO levels in irradiated PC3 cells (FIG. 5B left panel). In summary, these data confirmed that KmUTAG-fl was an effective reagent to detect increased SUMO levels, especially in the cytosol of PC3 cancer cells after acute UV exposure.

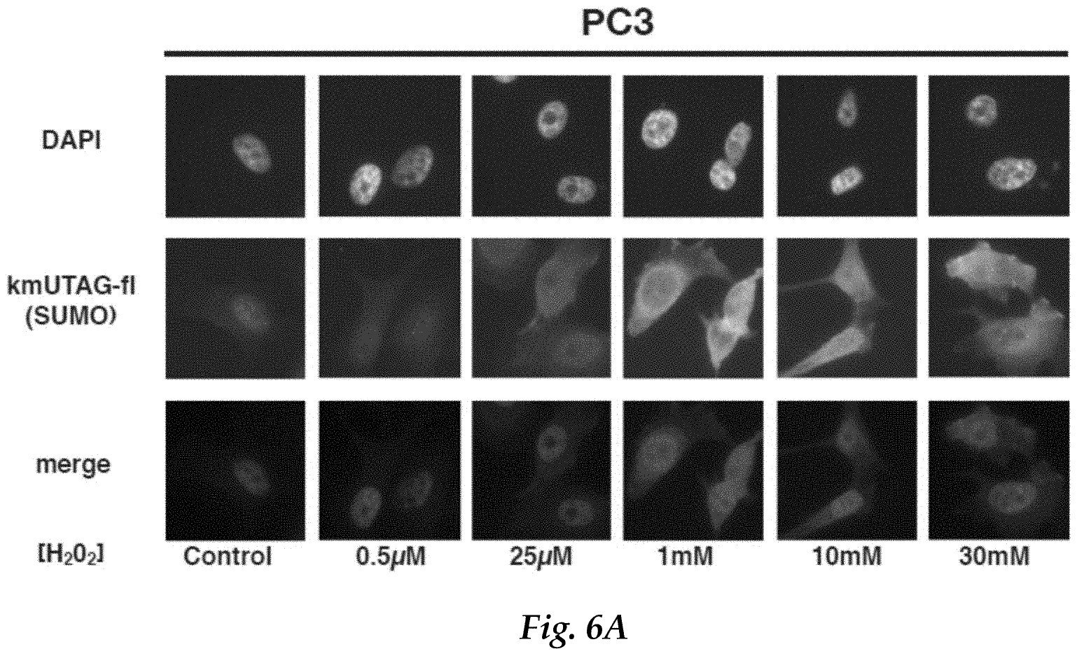

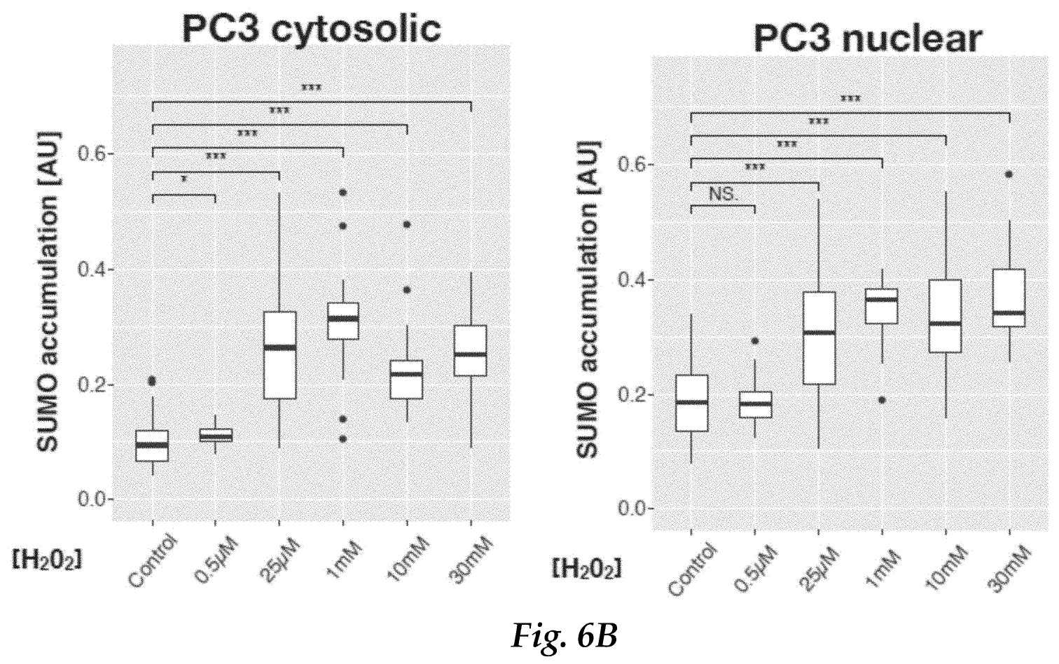

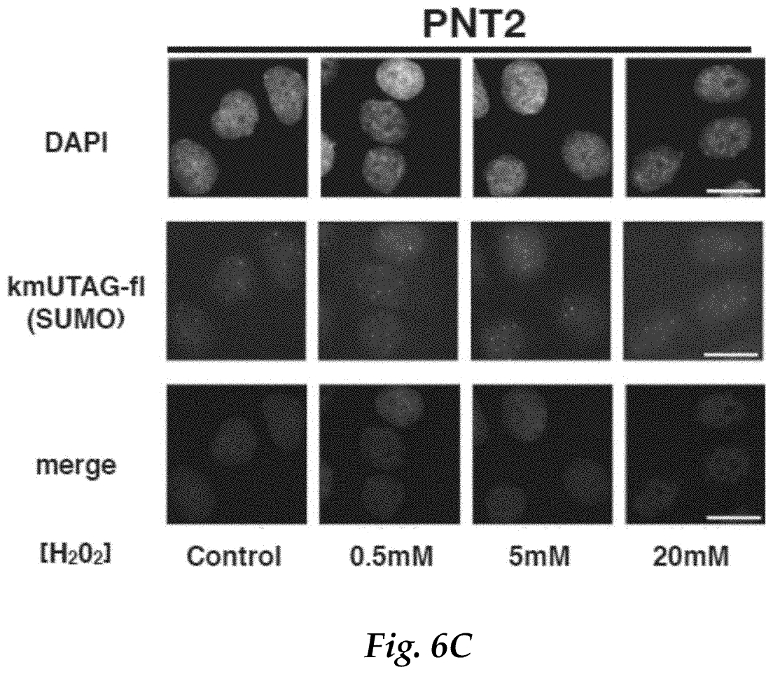

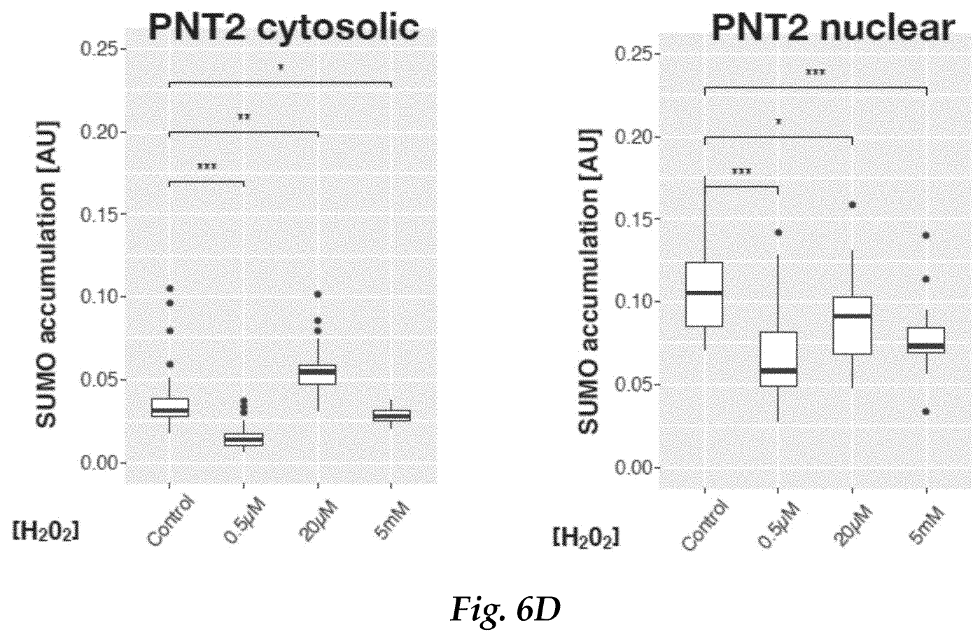

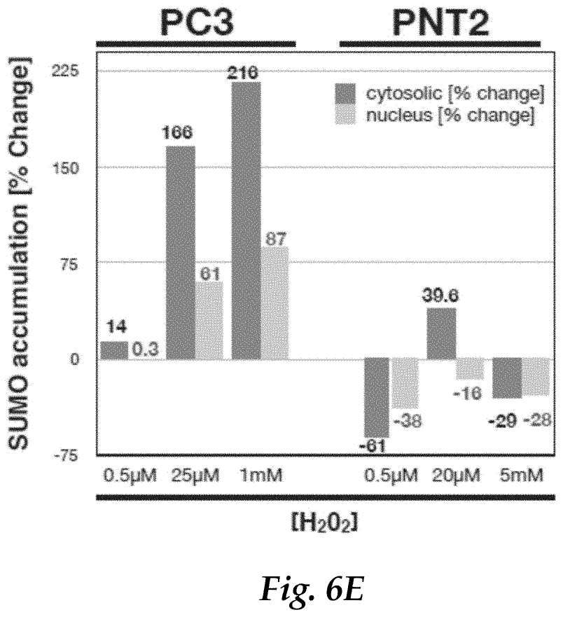

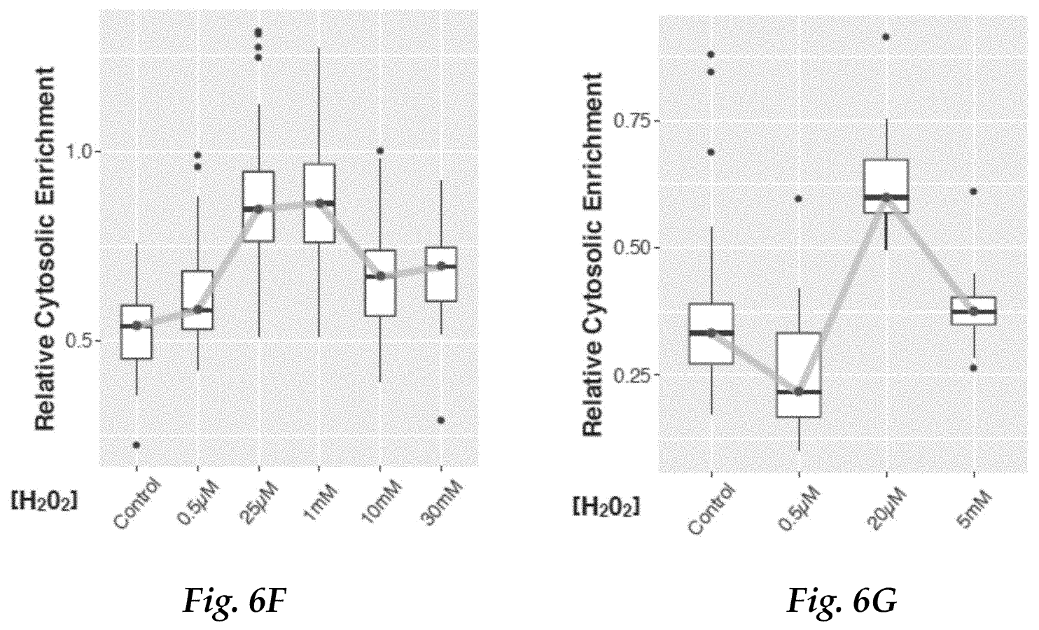

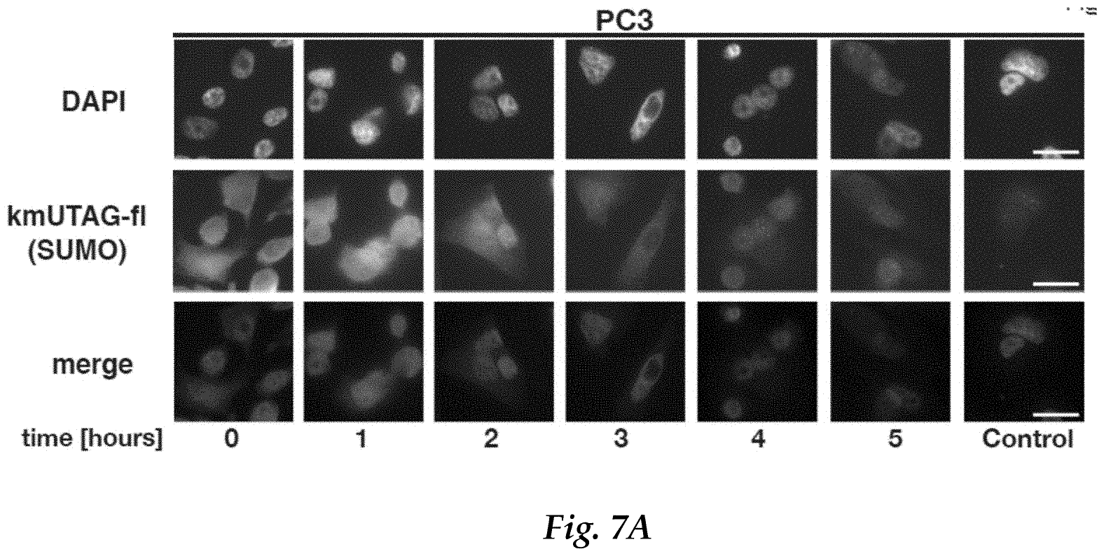

Cytosolic SUMO levels increased in PC3 prostate cancer cells due to oxidative stress. The rapid accumulation of extra-nuclear SUMO levels due to UV-irradiation prompted the investigation of the differences in SSR between PC3 and PNT2 cells after exposure to oxidative stress. PC3 and PNT2 cells were treated with varying concentrations of hydrogen peroxide [0.5 μM-30 mM H₂0₂] for 30 min, and immediately fixed and stained with KmUTAG-fl. Analysis of PC3 cells revealed a significant increase of cytosolic and nuclear KmUTAG-fl signal after treatment with 25 μM to 30 mM H₂0₂ (FIGS. 6A and 6B). The most significant median increase of SUMO staining intensity in PC3 cells was observed after 1 mM H₂0₂ treatment both in the cytosol (216%) and nucleus (87%) (FIGS. 6B and 6E). By comparison, the cytosolic and nuclear KmUTAG-fl signal in PNT2 cells did not reveal a steady trend of increasing SUMO signal following treatment (FIGS. 6C and 6D). Rather, statistically significant cytosolic PNT2 SUMO staining intensity increased after treatment with 20 μM H₂0₂ (˜40%) while cytosolic and nuclear SUMO accumulation fell below the intensity of the untreated control (28% reduction at 5 mM H₂0₂) (FIGS. 6D and 6E). Taking into account trends of both nuclear and cytosolic KmUTAG signals, we find an increasing RCE of SUMO after H₂0₂ treatment that is specific for PC3 cells (FIG. 5D, top panel).

Simultaneously, nuclear SUMO foci in nuclei of H₂0₂ treated PNT2 and PC3 cells were quantitated. Nuclear foci of SUMO2/3 are a hallmark of SUMO-specific localization in mammalian cells and co-localize with a variety of nuclear proteins including the PML protein. PML nuclear bodies are implicated in nuclear stress response and reported to increase over time when cells are exposed to genotoxic stressors (Liu, Shen, Guo, Cao, & Xu, 2017). In summary, the data suggest that both UV irradiation and oxidative stress resulted in a rapid and concentration-dependent accumulation of SUMO in PC3 cancer cells, especially in the cytosol. These differences were not observed under similar conditions in PNT2 cells. Therefore, the inventors focused on the novel stress-induced accumulation of SUMO conjugates specifically in the cytosol.

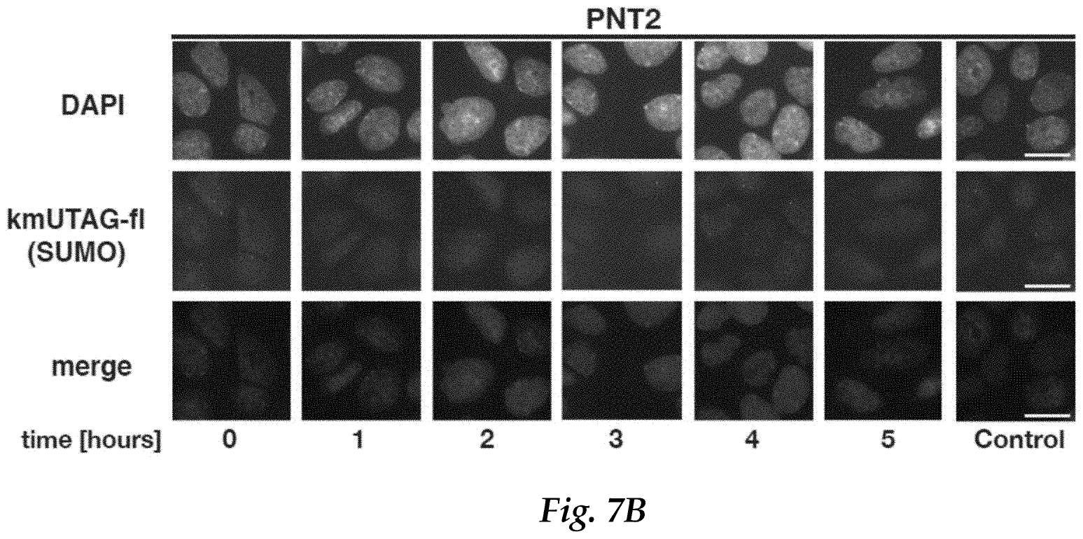

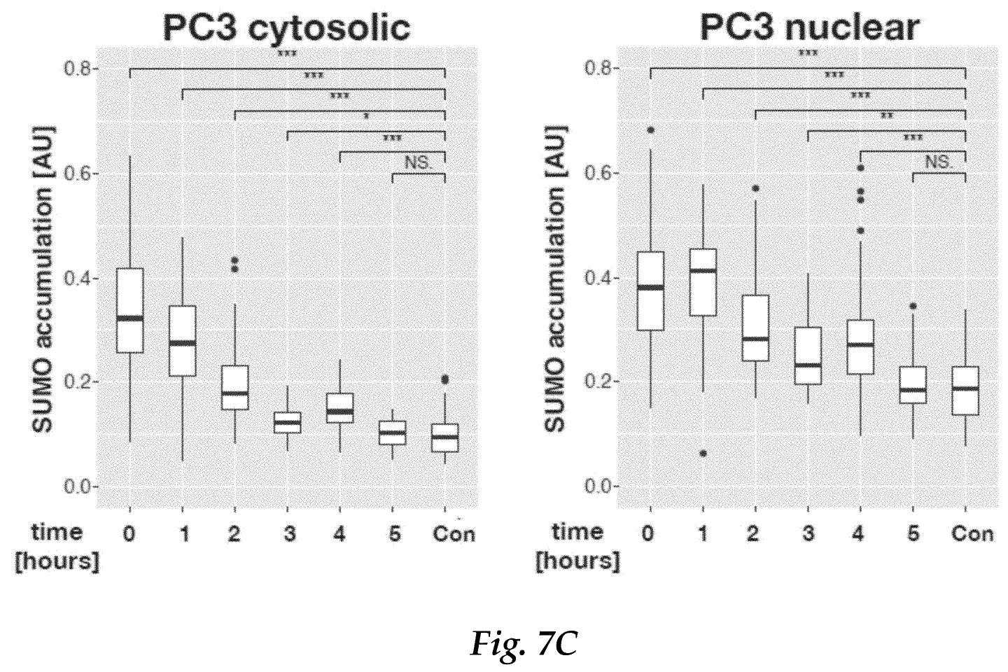

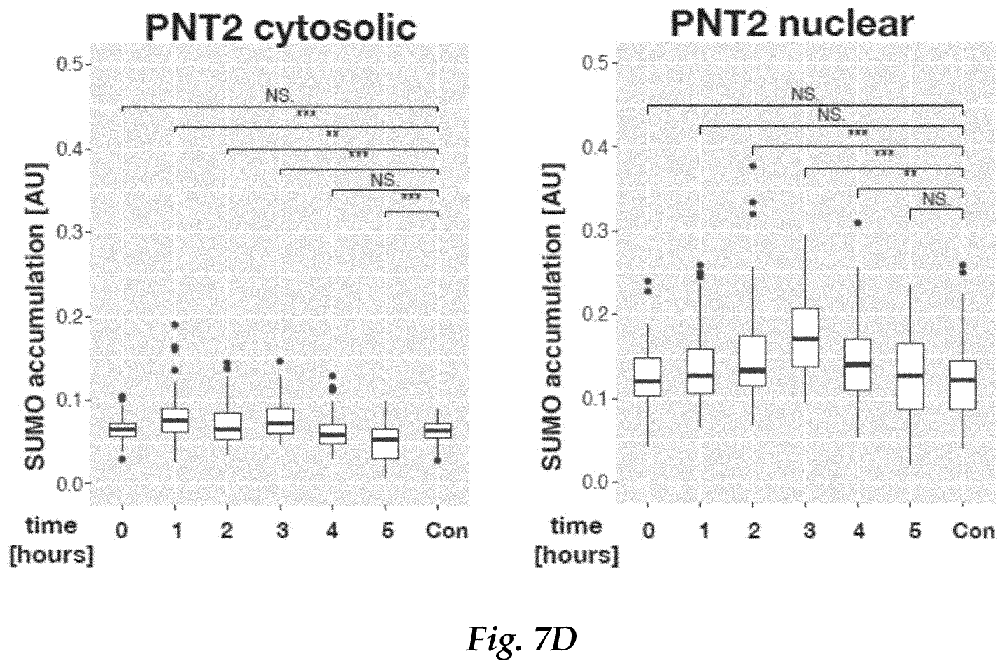

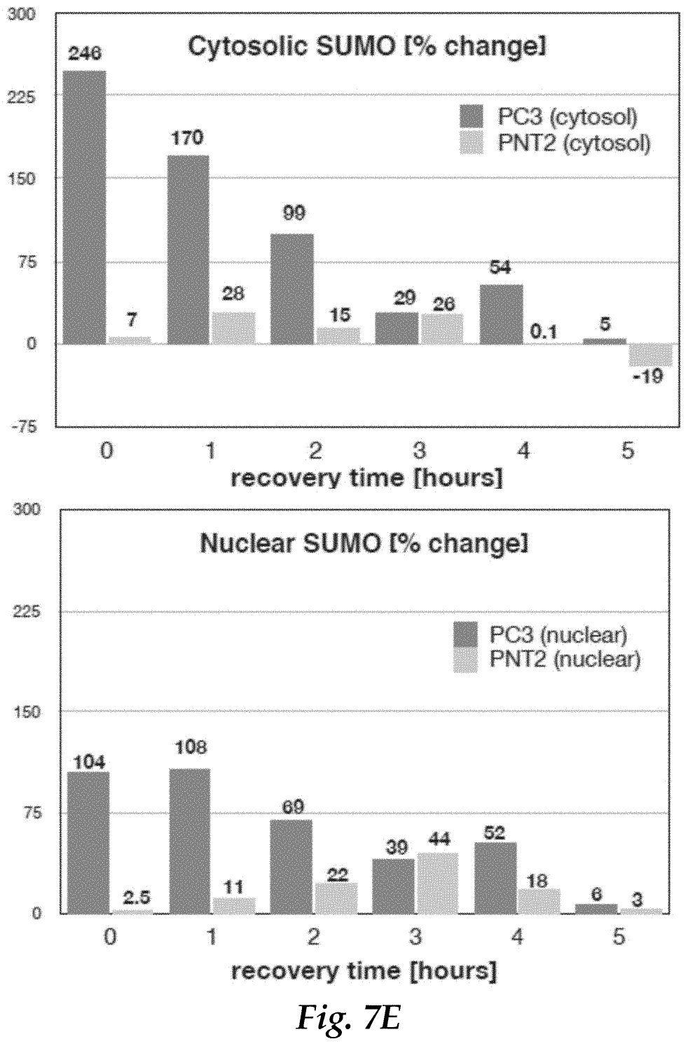

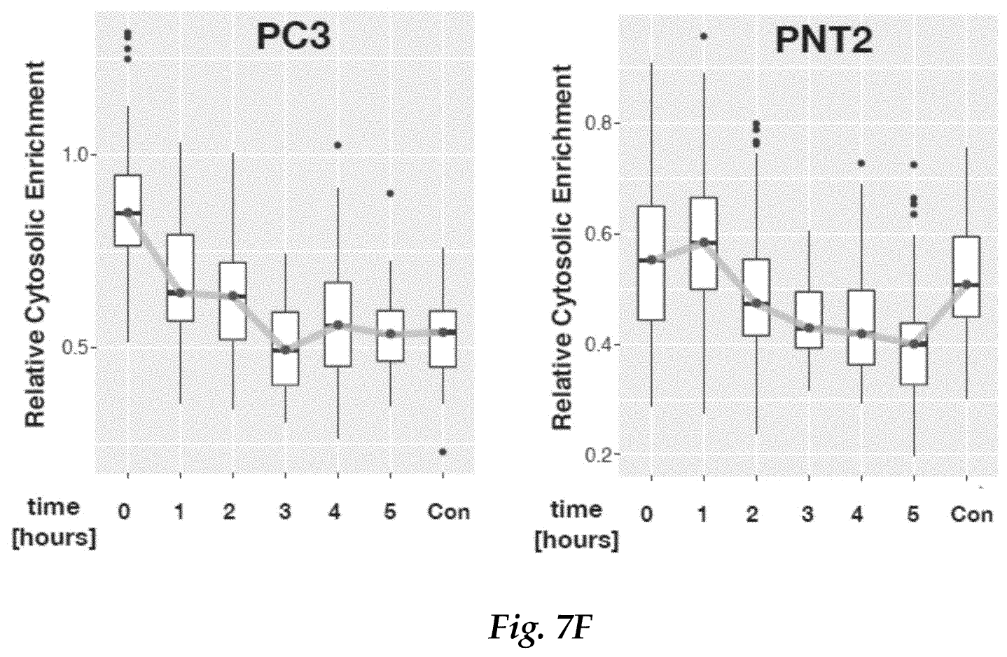

Recovery from peroxide-stress was accompanied by the reduction of cytosolic SUMO levels. The possibility that the rapid increase of cytosolic SUMO levels in PC3 cancer cells was a reversible process was investigated. PC3 and PNT2 cells were treated for 30 min with H₂0₂ before recovery in fresh media for 1 to 5 hours. As before, treatment with 1 mM peroxide resulted in a significant and rapid increase of cytosolic (161%) and nuclear (61%) SUMO signals in PC3 cells (FIGS. 7A and 7B). Remarkably, the nuclear SUMO signal of PC3 cells increased further and peaked after 1 hour (108%) into the recovery period (FIGS. 7C and 7E, right top panel). During recovery, a significant reduction of cytosolic and nuclear SUMO levels was apparent after 2 hours and SUMO levels returned to pre-treatment levels after 5 hours (FIGS. 7C and 7E top panels). Taking into account nuclear and cytosolic KmUTAG signals, a robust RCE of SUMO that decreased steadily during recovery (FIG. 7F left panel) was observed. These data indicated a rapid and dynamic fluctuation of SUMO levels in the nucleus and cytosol of PC3 cells as they responded to and recovered from oxidative stress.

In contrast, the increase of cytosolic (28%) and nuclear (44%) SUMO levels in PNT2 cells was significantly less pronounced (compare FIGS. 7D and 7E top and bottom panels). Notably the cytosolic and the nuclear SUMO signal of PNT2 cells peaked between 1 and 3 hours into the recovery period, suggesting slower SSR response kinetics in PNT2 cells (FIGS. 7D and 7E). This was also apparent when comparing the RCE of PC3 and PNT2 cells. The RCE of SUMO in PC3 cells peaked immediately after H₂0₂ treatment (0.85) while the RCE of SUMO in PNT2 cells peaked after 1 hour (0.58, FIG. 7F). In summary, these data suggest that SUMO levels of the PC3 prostate cancer cell line significantly increased after H₂0₂-induced stress and subsequently decreased as part of a recovery process.

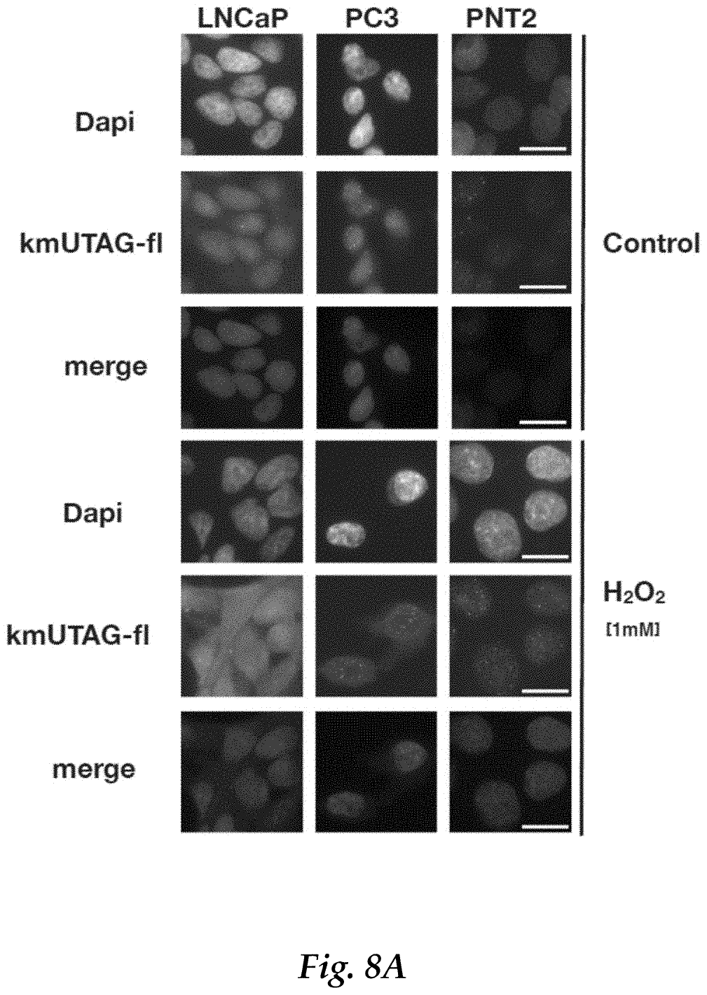

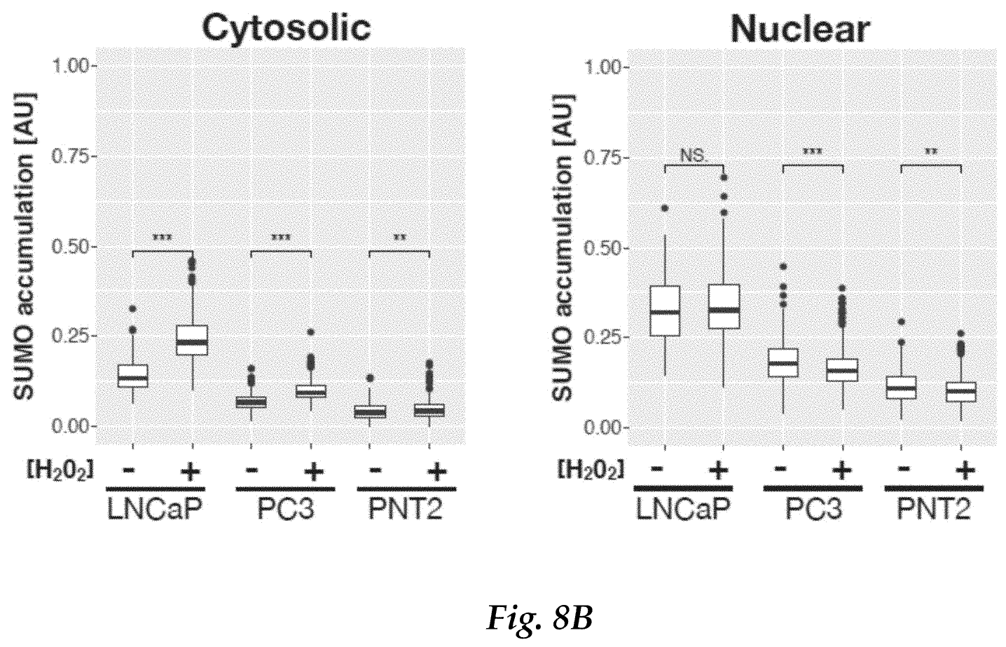

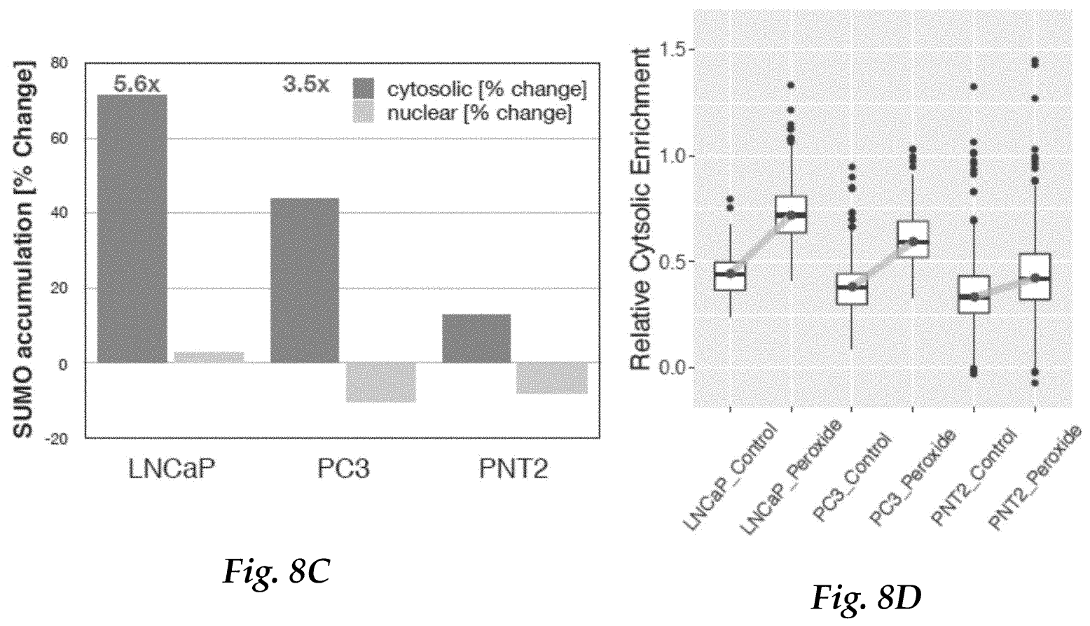

Stress-induced increase of cytosolic SUMO levels was observed in LNCaP prostate cancer cells with low metastatic potential. The finding that stress rapidly increased cytosolic SUMO levels in the highly aggressive PC3 cell line prompted the question as to whether this accumulation of SUMO is linked to metastatic potential. To assess this possibility, stress-induced SUMO levels in the LNCaP cell line possessing low metastatic potential, was compared to the highly aggressive PC3 and the non-malignant PNT2 cells (Spans et al., 2014). For this experiment, LNCaP, PC3, and PNT2 cells were simultaneously treated with H₂0₂ (1%, 30 min) as detailed above (FIGS. 5 and 6) and cytosolic and nuclear SUMO levels were compared before and after treatment. SUMO levels in nuclei and cytosol of untreated LNCaP, PC3, PNT2 cells showed different levels of SUMO before treatment (FIG. 8A). After H₂0₂ treatment, cytosolic SUMO levels of all cell lines increased significantly (FIG. 8B left). The largest increase in cytosolic SUMO signal after peroxide exposure was detected in LNCaP cells (71%), followed by PC3 (44%), and lastly PNT2 cells (13%) (FIGS. 8B and 8C). Therefore, cytosolic SUMO levels in LNCaP cells and in PC3 cells were 5.5 and 3.5-fold higher respectively than PNT2 cells. As before, a small but significant change in nuclear SUMO levels was detected in PC3 and PNT2 cell lines, albeit SUMO levels were decreased by 10.3% and 7.8% below the untreated controls samples, respectively (FIG. 7C). Concomitantly, H₂0₂ treated LNCaP cells did not show a significant change in nuclear SUMO levels (FIG. 8B right). Reduced nuclear SUMO levels after peroxide exposure were likely an authentic effect, as a large number of cells (n>300 for each) were scored for this comparison. Importantly, taking into account nuclear and cytosolic KmUTAG signals, a robust increase of the RCE of SUMO was detected for both LNCaP and PC3 cells (64% increase for LNCaP and 55%—increase for PC3 cells) but this change was much less pronounced in normal PNT2 cells (27% increase) (FIG. 8D). Importantly, the RCE of PC3 and LNCaP cells showed similar slopes and magnitude, suggesting that a difference in cancerous potential does not affect the observed cytosolic enrichment of SUMO. In summary, the data obtained with the KmUTAG-fl SUMO biosensor indicated that the detected increase in the RCE of SUMO levels were a hallmark of the SSR in cancerous cells.

Discussion The KmUTAG-fl biosensor reports on the presence and distribution of untagged, native SUMO conjugates in a variety of eukaryotic cells (Yin et al., 2019). Here, KmUTAG-fl was used to detect, quantitate, and analyze the cellular distribution of SUMO conjugates before and after exposure to acute proteotoxic and genotoxic stress. Using this approach, significant differences in the distribution of cytosolic (extra-nuclear) SUMO was detected between a normal (PNT2) and two cancer cell lines (PC3 and LNCaP). While the increase in cellular SUMO conjugate levels in response to stress has previously been observed, it is unknown how the SSR propagates throughout the cell or the extent of extra-nuclear SUMO distribution changes when cells undergo acute stress. In this study, a significant increase in cytosolic SUMO in response to oxidative and UV-irradiation stress was observed within 30 min of exposure, which was dependent on the concentration of the stressor and the time after stress exposure. Additionally, after recovery cytosolic SUMO levels returned to normal levels within a 5-hour interval. Nuclear SUMO levels were also altered during the SSR but overall the amplitude of this stress-induced modulation was lower or not significantly changed in the cell lines tested. The increase of cytosolic SUMO in response to peroxide treatment ranged from 12.6-70.6% (average ˜5-fold) but was statistically significant for both PC3 and LNCaP cell lines when compared to PNT2 cells (Unpaired t-test), indicating this stress induced effect was consistent and reproducible. Overall, the findings demonstrated that the SUMO biosensor KmUTAG-fl could differentiate between the SSR of cancerous and normal cells. Additionally, the results offer new insights into the dynamics of the SSR and how cancer cells modulate SUMO levels in response to stress.

Cancer cells are known to modulate their SUMO dynamics as part of a strategy to become more stress-tolerant (Seeler & Dejean, 2017). One reason is that cancer cells are under constant threat of adverse conditions, including hypoxia within tumors, immune invasions, and aneuploidies that threaten protein homeostasis (Muz, la Puente, Azab, & Azab, 2015; Oromendia & Amon, 2014). Thus, cancer cells require enhanced stress response pathways to mitigate these effects and to maintain proteostasis and genome integrity. For this study, three different cell lines were used to observe the SSR and to identify unique cell-type specific features. PNT2 cells were established by SV40-mediated immortalization of normal adult prostatic epithelial cells; PC3 cells represent an adenocarcinoma cell line with high metastatic potential; and the LNCaP cell line has low metastatic potential. These human-derived prostate cell lines do not only differ in their tumorigenicity but also their chromosomal make-up. PNT2 cells are non-malignant normal prostate epithelium immortalized with SV40; PC3 have 62 chromosomes (Tai et al., 2011); and LNCaP harbor 79-91 chromosomes (Horoszewicz et al., 1983). Therefore, both the expression levels and copy number of SUMO genes are increased in these tumorigenic cell lines (Kerscher unpublished results). The observation that cytosolic SUMO levels are significantly increased after stress exposure of PC3 and LNCaP cells can be due to increased expression levels of SUMO in comparison to PNT2 cells.

Considering that sumoylation is considered a predominantly nuclear event, the rapid stress-induced increase and decrease of SUMO conjugates in the cytosol of cancer cells is an interesting finding. A recent review on sub-cellular sumoylation in the heart posits that sumoylation in the extra-nuclear compartment of cardiomyocytes is generally cardio-protective (Le, Martin, Fujiwara, & Abe, 2017). More specific, results from a study on SUMO2/3 suggest that stress-induced sumoylation serves to temporarily stabilize (keep soluble) misfolding proteins and targets those that can't be refolded for proteasomal degradation (Liebelt et al., 2019). Nevertheless, most information on stress-induced sumoylation concerns its nuclear effects and ignores the cytoplasmic roles of SUMO. For example, SUMO isoforms may serve a chaperone-like function to maintain the homeostasis of large chromatin-associated nuclear proteins during stress (Seifert et al., 2015) and stress-induced sumoylation is required for transcriptional re-programming (Lewicki et al., 2015). In this respect, the observation of a transient increase of SUMO due to acute UV and oxidative stress underscores the dynamic nature of SSR in the cell. Additionally, it is an important reminder that the effects of SUMO (and especially the SSR in normal and transformed cell lines) are likely to produce very different, cell line-specific outcomes. Many proteomics studies on the SSR in mammalian cell lines are conducted in cancer-derived cell lines and these studies do not always include normal, immortalized comparators.

In summary, using the pan SUMO-specific KmUTAG-fl biosensor, a transient, reversible, stress-induced increase of SUMO conjugates has been identified in the cytosol of PC3 and LNCaP cells. This SUMO enrichment clearly distinguishes PC3 and LNCaP cells from normal immortalized PNT2 cells, suggesting that it may be part of a stress tolerance pathway that is specific for cancer cells.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

Aurich-Costa, J., Vannier, A., Grégoire, E., Nowak, F., & Cherif, D. (2001). IPM-FISH, a new M-FISH approach using IRS-PCR painting probes: application to the analysis of seven human prostate cell lines. Genes, Chromosomes & Cancer, 30(2), 143-160.
Baker, M. (2015). Reproducibility crisis: Blame it on the antibodies. Nature News, 521(7552), 274-276. http://doi.org/10.1038/521274a
Berthon, P., Cussenot, O., Hopwood, L., Leduc, A., & Maitland, N. (1995). Functional expression of sv40 in normal human prostatic epithelial and fibroblastic cells—differentiation pattern of nontumorigenic cell-lines. International Journal of Oncology, 6(2), 333-343. http://doi.org/10.3892/ijo.6.2.333
Bossis, G., & Melchior, F. (2006). Regulation of SUMOylation by Reversible Oxidation of SUMO Conjugating Enzymes. Molecular Cell, 21(3), 349-357. http://doi.org/10.1016/j.molcel.2005.12.019
Elmore, Z. C., Donaher, M., Matson, B. C., Murphy, H., Westerbeck, J. W., & Kerscher, O. (2011). Sumo-dependent substrate targeting of the SUMO protease Ulp1. BMC Biology, 9, 74-74. http://doi.org/10.1186/1741-7007-9-74
Golebiowski, F., Matic, I., Tatham, M. H., Cole, C., Yin, Y., Nakamura, A., et al. (2009). System-wide changes to SUMO modifications in response to heat shock. Science Signaling, 2(72), ra24-ra24. http://doi.org/10.1126/scisignal.2000282
Hay, R. T. (2005). SUMO: a history of modification. Molecular Cell, 18(1), 1-12. http://doi.org/10.1016/j.molcel.2005.03.012
Hendriks, I. A., D'Souza, R. C. J., Yang, B., Verlaan-de Vries, M., Mann, M., & Vertegaal, A. C. O. (2014). Uncovering global SUMOylation signaling networks in a site-specific manner. Nature Structural & Molecular Biology, 21(10), 927-936. http://doi.org/10.1038/nsmb.2890
Horoszewicz, J. S., Leong, S. S., Kawinski, E., Karr, J. P., Rosenthal, H., Chu, T. M., et al. (1983). LNCaP model of human prostatic carcinoma. Cancer Research, 43(4), 1809-1818.
Karami, S., Lin, F.-M., Kumar, S., Bahnassy, S., Thangavel, H., Quttina, M., et al. (2017). Novel SUMO-Protease SENP7S Regulates β-catenin Signaling and Mammary Epithelial Cell Transformation. Scientific Reports, 7(1), 46477. http://doi.org/10.1038/srep46477
Kerscher, O., Felberbaum, R., & Hochstrasser, M. (2006). Modification of proteins by ubiquitin and ubiquitin-like proteins. Cell and Developmental Biology, 22, 159-180. http://doi.org/10.1146/annurev.cellbio.22.010605.093503
Kerscher, O. (2016). SUMOylation (pp. 1-11). eLS. John Wiley & Sons, Ltd: Chichester. http://doi.org/10.1002/9780470015902.a0021849.pub2
Knipscheer, P., van Dijk, W. J., Olsen, J. V., Mann, M., & Sixma, T. K. (2007). Noncovalent interaction between Ubc9 and SUMO promotes SUMO chain formation. EMBO Journal, 26(11), 2797-2807. http://doi.org/10.1038/sj.emboj.7601711
Le, N.-T., Martin, J. F., Fujiwara, K., & Abe, J.-I. (2017). Sub-cellular localization specific SUMOylation in the heart. Biochimica Et Biophysica Acta. Molecular Basis of Disease, 1863(8), 2041-2055. http://doi.org/10.1016/j.bbadis.2017.01.018
Lewicki, M. C., Srikumar, T., Johnson, E., & Raught, B. (2015). The S. cerevisiae SUMO stress response is a conjugation-deconjugation cycle that targets the transcription machinery. Journal of Proteomics, 118, 39-48. http://doi.org/10.1016/j.jprot.2014.11.012
Liebelt, F., Sebastian, R. M., Moore, C. L., Mulder, M. P. C., Ovaa, H., Shoulders, M. D., & Vertegaal, A. C. O. (2019). SUMOylation and the HSF1-Regulated Chaperone Network Converge to Promote Proteostasis in Response to Heat Shock. Cell Reports, 26(1), 236-249.e4. http://doi.org/10.1016/j.celrep.2018.12.027
Liu, S.-B., Shen, Z.-F., Guo, Y.-J., Cao, L.-X., & Xu, Y. (2017). PML silencing inhibits cell proliferation and induces DNA damage in cultured ovarian cancer cells. Biomedical Reports, 7(1), 29-35. http://doi.org/10.3892/br.2017.919 Mattoscio, D. (2015). The SUMO conjugating enzyme UBC9 as a biomarker for cervical HPV infections. Ecancermedicalscience, 9. http://doi.org/10.3332/ecancer.2015.534
McQuin, C., Goodman, A., Chernyshev, V., Kamentsky, L., Cimini, B. A., Karhohs, K. W., et al. (2018). CellProfiler 3.0: Next-generation image processing for biology. PLoS Biology, 16(7), e2005970. http://doi.org/10.1371/journal.pbio.2005970
Muz, B., la Puente, de, P., Azab, F., & Azab, A. K. (2015). The role of hypoxia in cancer progression, angiogenesis, metastasis, and resistance to therapy. Hypoxia (Auckland, N.Z.), 3, 83-92. http://doi.org/10.2147/HP.S93413
Muench, S. P., Rafferty, J. B., Mcleod, R., Rice, D. W., & Prigge, S. T. (2003). Expression, purification and crystallization of the Plasmodium falciparum enoyl reductase. Acta Cryst. D59, 1246-1248.
Oromendia, A. B., & Amon, A. (2014). Aneuploidy: implications for protein homeostasis and disease. Disease Models & Mechanisms, 7(1), 15-20. http://doi.org/10.1242/dmm.013391
Peek, J., Harvey, C., Gray, D., Rosenberg, D., Kolla, L., Levy-Myers, R., et al. (2018). SUMO targeting of a stress-tolerant Ulp1 SUMO protease. PLoS ONE, 13(1), e0191391. http://doi.org/10.1371/journal.pone.0191391
Pelisch, F., Tammsalu, T., Wang, B., Jaffray, E. G., Gartner, A., & Hay, R. T. (2017). A SUMO-Dependent Protein Network Regulates Chromosome Congression during Oocyte Meiosis. Molecular Cell, 65(1), 66-77. http://doi.org/10.1016/j.molcel.2016.11.001
Pinto, M. P., Carvalho, A. F., Grou, C. P., Rodriguez-Borges, J. E., Sá-Miranda, C., & Azevedo, J. E. (2012). Heat shock induces a massive but differential inactivation of SUMO-specific proteases. Biochimica Et Biophysica Acta, 1823(10), 1958-1966. http://doi.org/10.1016/j.bbamcr.2012.07.010
Saitoh, H., & Hinchey, J. (2000). Functional heterogeneity of small ubiquitin-related protein modifiers SUMO-1 versus SUMO-2/3. The Journal of Biological Chemistry, 275(9), 6252-6258.
Salerno, J. C., Ngwa, V. M., Nowak, S. J., Chrestensen, C. A., Healey, A. N., & McMurry, J. L. (2016). Novel cell penetrating peptide-adaptors effect intracellular delivery and endosomal escape of protein cargos. Journal of Cell Science. http://doi.org/10.1242/jcs.182113
Sarge, K. D., & Park-Sarge, O.-K. (2009). Sumoylation and human disease pathogenesis. Trends in Biochemical Sciences, 34(4), 200-205. http://doi.org/10.1016/j.tibs.2009.01.004
Seeler, J.-S., & Dejean, A. (2017). SUMO and the robustness of cancer. Nature Reviews Cancer, 17(3), 184-197. http://doi.org/10.1038/nrc.2016.143
Seifert, A., Schofield, P., Barton, G. J., & Hay, R. T. (2015). Proteotoxic stress reprograms the chromatin landscape of SUMO modification. Science Signaling, 8(384), rs7-rs7. http://doi.org/10.1126/scisignal.aaa2213
Spans, L., Helsen, C., Clinckemalie, L., Van den Broeck, T., Prekovic, S., Joniau, S., et al. (2014). Comparative genomic and transcriptomic analyses of LNCaP and C4-2B prostate cancer cell lines. PLoS ONE, 9(2), e90002. http://doi.org/10.1371/journal.pone.0090002
Srikumar, T., Lewicki, M. C., Costanzo, M., Tkach, J. M., van Bakel, H., Tsui, K., et al. (2013). Global analysis of SUMO chain function reveals multiple roles in chromatin regulation. The Journal of Cell Biology, 201(1), 145-163. http://doi.org/10.1083/jcb.201210019.dv
Tai, S., Sun, Y., Squires, J. M., Zhang, H., Oh, W. K., Liang, C.-Z., & Huang, J. (2011). PC3 is a cell line characteristic of prostatic small cell carcinoma. The Prostate, 71(15), 1668-1679. http://doi.org/10.1002/pros.21383
Wang, Q., Xia, N., Li, T., Xu, Y., Zou, Y., Zuo, Y., et al. (2013a). SUMO-specific protease 1 promotes prostate cancer progression and metastasis. Oncogene, 32(19), 2493-2498. http://doi.org/10.1038/onc.2012.250
Wang, R.-T., Zhi, X.-Y., Zhang, Y., & Zhang, J. (2013b). Inhibition of SENP1 induces radiosensitization in lung cancer cells. Experimental and Therapeutic Medicine, 6(4), 1054-1058. http://doi.org/10.3892/etm.2013.1259
Wang, Z., & Prelich, G. (2009). Quality control of a transcriptional regulator by SUMO-targeted degradation. Molecular and Cellular Biology, 29(7), 1694-1706. http://doi.org/10.1128/MCB.01470-08
Wu, F., Zhu, S., Ding, Y., Beck, W. T., & Mo, Y.-Y. (2009). MicroRNA-mediated regulation of Ubc9 expression in cancer cells. Clinical Cancer Research, 15(5), 1550-1557. http://doi.org/10.1158/1078-0432.CCR-08-0820
Yin, R., Harvey, C., Shakes, D. C., & Kerscher, O. (2019). Localization of SUMO-modified Proteins Using Fluorescent Sumo-trapping Proteins. Journal of Visualized Experiments: JoVE, (146), e59576. http://doi.org/10.3791/59576
Zhang, X.-D., Goeres, J., Zhang, H., Yen, T. J., Porter, A. C. G., & Matunis, M. J. (2008). SUMO-2/3 modification and binding regulate the association of CENP-E with kinetochores and progression through mitosis. Molecular Cell, 29(6), 729-741. http://doi.org/10.1016/j.molcel.2008.01.013

1. A method of detecting one or more small ubiquitin-like modifier protein (SUMO)-modified proteins in a biological sample, wherein the method comprises:
combining the biological sample with a protein comprising a pan-Sumo trapping protein, wherein the pan-Sumo trapping protein is stress-tolerant and binds Sumo-modified proteins with high avidity; and
detecting one or more SUMO-modified proteins and wherein the biological sample comprises an in vitro sample of cells or wherein the biological sample comprises in vivo cells.

2. The method of claim 1, wherein stress-tolerant comprises being resistant to one or more of elevated temperatures, reducing agents, denaturants, oxidizing agents, and non-ionic detergents, and pro-longed incubation time.

3. The method of claim 1, wherein the pan-Sumo trapping protein comprises an inactive C-terminal catalytic domain of a SUMO protein and is more resistant to oxidative stress when bound to the one or more SUMO-modified proteins as compared to an active C-terminal catalytic domain of a corresponding SUMO protease.

4. The method of claim 3, wherein the pan-SUMO trapping protein comprises a cysteine to serine mutation at the amino acid corresponding to amino acid 517 of the amino acid sequence of Ulp1 Kluyveromyces marxianus (Km).

5. The method of claim 4, wherein the inactive C-terminal catalytic domain comprises an amino acid sequence comprising at least 65%, 70%, 75%, 80%, 82%, 85%, 86%, 87%, 88%, 89%, 90%, 91%, 92%, 93%, 94%, 95%, 96%, 97%, 98%, 99%, or 100% sequence identity with the amino acid sequence of the UD domain of KmUTAG or SEQ ID NO: 2.

6. The method of claim 1, wherein the pan-Sumo trapping protein comprises an amino acid sequence as set forth in SEQ ID NO: 2.

7. The method of claim 1, wherein the pan-Sumo trapping protein comprises a fusion protein.

8. The method of claim 7, wherein the fusion protein comprises a fluorescent protein and/or a purification tag.

9. The method of claim 8, wherein the fluorescent (fl) protein comprises mCherry, mPlum, mRaspberry, HcRed-Tandem, mRFP1, mApple, mRuby, mStrawberry, mTangerine, DsRed-Monomer, TagRFP-T, mOrange, dTomatoTandem, Kusabira Orange, mBanana, TagYFP, TagCFP, mCitrine, mECFP, mTagBFP, or mWasabi.

10. The method of claim 8, wherein the fusion protein comprises amino acid sequence SEQ ID NO: 4.

11. The method of claim 1, wherein the pan-Sumo trapping protein is encoded by SEQ ID NO: 1.

12. The method of claim 7, wherein the fusion protein is encoded by SEQ ID NO: 3.

13. The method of claim 1, wherein the pan-Sumo trapping protein comprises a label and optionally wherein the label comprises biotin, enzyme, fluorescent label, chemiluminescent label, a radioactive label, or a calorimetric label.

14. The method of claim 1, wherein the method of detecting one or more SUMO-modified proteins comprises quantitating the one or more SUMO-modified proteins.

15. The method of claim 14, wherein the method further comprises diagnosing or monitoring a disease, and optionally wherein a change in the quantity of one or more SUMO-modified proteins indicate presence of a disease, progression of a disease, or alleviation of a disease.

16. The method of claim 15, wherein the cells are diseased cells, and wherein optionally the diseased cells comprise cells with genotoxic and/or proteotoxic stress.

17. The method of claim 16, wherein the diseased cells comprise cancer cell, and optionally wherein the cancer cells comprise cells from prostate cancer, breast cancer, cervical cancer, ovarian cancer, lung cancer, ovarian cancer, pancreatic cancer, colorectal cancer, bladder cancer, lymphoma, skin cancer, stomach cancer, liver cancer, leukemia (blood cancer), or solid tumor cancer.

18. The method of claim 16, wherein the diseased cells comprise cells under oxidative stress, and optionally wherein the oxidative stress causes Alzheimer's disease, Parkinson's disease, amyotrophic lateral sclerosis, and other neurodegenerative disease, arthritis, asthma, Crohn's disease, irritable bowel syndrome, ulcerative colitis, cardiovascular diseases, or autoimmune diseases.

19. The method of claim 16, wherein the diseased cells comprise cells from an infection, and optionally wherein the infection is a bacterial or viral infection.

20. The method of claim 15, wherein the method comprises diagnosing a disease by detecting one or more SUMO biomarkers for the disease.

21. The method of claim 1, wherein the biological sample comprises biological fluid or tissue sample from a subject, and optionally wherein the biological fluid comprises urine, blood, blood serum, plasma, bile, fecal aspirate, intestinal aspirate, cerebrospinal fluid, or saliva and tissue sample comprises sample from a swab or a biopsy.


SEQUENCE LISTING (4 sequences)

SEQ ID NO: 1 - DNA, 645 bp, Artificial Sequence
  kmUTAG
atccccgaactgagctccaaggacgtggaagccgtgaaggccaccctgaggaggagcgacaacagcgtgctgagcagcaagtataccctggaggtgagcgtcagggacttcaagaccctggcccctaacaggtggctgaacgacaccatcatcgagttcttcatgaaatacatcgagaacaacacccccaagaccgtggccttcaactccttcttttacagcaccctggccaacaggggataccagggcgtgaggaggtggatgaagaggaagaaggtcgacatcctggacctggaaaggatcttcatccccgtgaacctcaacgacagccactggaccctgggcatcatcgatatcaagaacaagaggatcctgtacctggactccctgagctccggcgccaacagcgtgagcttcctgatcatgaagaacatccagagctacctgatcgaggagtccaagaacaagctgggcaaggactttgagctgtgccacctggactgccctcagcagcccaacggcagcgatagcggcatctacgtgtgcctgaacaccctgtacatgagcaagaactacagcctggacttcaatgcccaggacgccgtgaacatgagggtgtacatcggccatctgatcctgagcaag

SEQ ID NO: 2 - protein, 215 aa, Artificial Sequence
  kmUTAG
IPELSSKDVEAVKATLRRSDNSVLSSKYTLEVSVRDFKTLAPNRWLNDTIIEFFMKYIENNTPKTVAFNSFFYSTLANRGYQGVRRWMKRKKVDILDLERIFIPVNLNDSHWTLGIIDIKNKRILYLDSLSSGANSVSFLIMKNIQSYLIEESKNKLGKDFELCHLDCPQQPNGSDSGIYVCLNTLYMSKNYSLDFNAQDAVNMRVYIGHLILSK

SEQ ID NO: 3 - DNA, 1485 bp, Artificial Sequence
  kmUTAG-fl fusion protein coding sequence
cgccgggttccttcccctctagaaataattttgtttactttaagaaggagatataccatgccggatcgcgtgcgcgcagtctctcactggagcagcggatccgaattcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgggccgagggccgccactccaccggcggcatggacgagctgtacaagtccggactcagatctcgagctatgatccccgaactgagctccaaggacgtggaagccgtgaaggccaccctgaggaggagcgacaacagcgtgctgagcagcaagtataccctggaggtgagcgtcagggacttcaagaccctggcccctaacaggtggctgaacgacaccatcatcgagttcttcatgaaatacatcgagaacaacacccccaagaccgtggccttcaactccttcttttacagcaccctggccaacaggggataccagggcgtgaggaggtggatgaagaggaagaaggtcgacatcctggacctggaaaggatcttcatccccgtgaacctcaacgacagccactggaccctgggcatcatcgatatcaagaacaagaggatcctgtacctggactccctgagctccggcgccaacagcgtgagcttcctgatcatgaagaacatccagagctacctgatcgaggagtccaagaacaagctgggcaaggactttgagctgtgccacctggactgccctcagcagcccaacggcagcgatagcggcatctacgtgtgcctgaacaccctgtacatgagcaagaactacagcctggacttcaatgcccaggacgccgtgaacatgagggtgtacatcggccatctgatcctgagcaag

SEQ ID NO: 4 - protein, 495 aa, Artificial Sequence
  kmUTAG-fl fusion protein
RRVPSPLEIILFTLRRRYTMPDRVRAVSHWSSGSEFMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAMIPELSSKDVEAVKATLRRSDNSVLSSKYTLEVSVRDFKTLAPNRWLNDTIIEFFMKYIENNTPKTVAFNSFFYSTLANRGYQGVRRWMKRKKVDILDLERIFIPVNLNDSHWTLGIIDIKNKRILYLDSLSSGANSVSFLIMKNIQSYLIEESKNKLGKDFELCHLDCPQQPNGSDSGIYVCLNTLYMSKNYSLDFNAQDAVNMRVYIGHLILSK